(19) United States
(12) Patent Application Publication
(10) Pub. No.: US 2026/0171245 A1
(43) Pub. Date: Jun. 18, 2026

(54) METHOD FOR PROVIDING INFORMATION FOR DISEASE DIAGNOSIS THROUGH DIR CONVERSION

(71) Applicant: KOREA RESEARCH INSTITUTE OF BIOSCIENCE AND BIOTECHNOLOGY, Daejeon (KR)

(72) Inventors: Hyun Woo OH, Daejeon (KR); Sang Woon SHIN, Daejeon (KR)

(73) Assignee: KOREA RESEARCH INSTITUTE OF BIOSCIENCE AND BIOTECHNOLOGY, Daejeon (KR)

(21) Appl. No.: 19/125,054

(22) PCT Filed: Nov. 2, 2023
(86) PCT No.: PCT/KR2023/017410

§ 371 Date: Apr. 28, 2025

(30) Foreign Application Priority Data
Nov. 2, 2022 (KR) 10-2022-0144736
Nov. 2, 2023 (KR) 10-2023-0149992

(51) Int. Cl. G16H 50/20; C12Q 1/6809; C12Q 1/6883; G16B 20/20; G16H 50/70

(52) U.S. Cl. CPC G16H 50/20; C12Q 1/6809; C12Q 1/6883; G16B 20/20; G16H 50/70

(57) ABSTRACT

An aspect relates to a method of finding an index gene in a genome equal to a DEG and performing a DEG-index-ratio (Dir) transformation using the index gene to enable RNA-seq data to be used to provide information for diagnosing a disease, and through a multidimensional Dir transformation according to an aspect controls individual variations in RNA-seq results and is useful in providing information for early diagnosis of a disease.

### TECHNICAL FIELD

The present disclosure relates to a method of providing information for diagnosing a disease by finding a particular index gene from RNA-seq data and performing a DEG-index-ratio (Dir) transformation.

### BACKGROUND ART

Currently, RNA-seq technology is cost-effective in diagnosing diseases by examining expressions of all genes at once compared to several types of other diagnostic techniques. However, the resulting data variations are severe, and thus, the RNA-seq technology is not currently and directly used for disease diagnoses, and for practical use, compositional data (gene counts or data normalized in various methods) secured by RNA-seq needs to be transformed. Data variations are estimated to be manifested not only by technical variations, but also by various environmental variations such as sleep, nutrition, hydration, and other stresses.

In RNA-seq, environmental factors as mentioned above act as limitations in applying RNA-seq data for disease diagnoses. Currently, data instability caused by technical variations such as sequencing errors may be overcome by known normalization methods to date. However, RNA-seq is not easily applied to disease diagnoses due to RNA-seq data instability caused by variations in various environmental differences between samples, and marker genes for particular diseases are not easily searched for due to variations.

Recently, deep learning methods have been proposed to normalize environmental variations by using generative adversarial networks (GANs). GAN algorithms have the potential to eliminate variations in RNA-seq data through multidimensional normalization. However, high prediction accuracy has not yet been achieved.

Accordingly, the present inventors have completed the present invention to provide methods of diagnosing diseases by transforming read number values of particular disease genes by using particular index genes as methods for removing environmental variations that always appear in RNA-seq.

### DISCLOSURE OF INVENTION

### Technical Problem

An aspect is to provide a method of providing information for diagnosing a disease including: selecting at least one particular index gene from among all genes by using a differentially expressed gene (DEG) differentially expressed in a disease state compared to a normal control group; performing a multidimensional DEG-index-ratio (Dir) transformation using the DEG and the index gene; and determining whether or not a difference between DEG expressions of the normal control group and the disease state is significant.

Another aspect is to provide a method of selecting a gene significant for a disease state including: selecting at least one particular index gene from among all genes by using a differentially expressed gene (DEG) differentially expressed in a disease state compared to a normal control group; performing a DEG-index-ratio (Dir) transformation using the DEG and the index gene; and determining whether or not a difference between DEG expressions of the normal control group and the disease state is significant.

Another aspect is to provide a method of diagnosing a disease state including: selecting at least one particular index gene from among all genes by using a differentially expressed gene (DEG) differentially expressed in a disease state compared to a normal control group; performing a DEG-index-ratio (Dir) transformation using the DEG and the index gene; and determining whether or not a difference between DEG expressions of the normal control group and the disease state is significant.

Another aspect is to provide a method of treating a disease state including: selecting at least one particular index gene from among all genes by using a differentially expressed gene (DEG) differentially expressed in a disease state compared to a normal control group; performing a DEG-index-ratio (Dir) transformation using the DEG and the index gene; and determining whether or not a difference between DEG expressions of the normal control group and the disease state is significant.

Another aspect is to provide a computer-readable recording medium having recorded thereon a program for causing a computer to execute a method of providing information for diagnosing a disease.

### Solution to Problem

An aspect provides a method of providing information for diagnosing a disease including selecting at least one particular index gene from among all genes by using a differentially expressed gene (DEG) differentially expressed in a disease state compared to a normal control group; performing a DEG-index-ratio (Dir) transformation using the DEG and the index gene; and determining whether or not a difference between DEG expressions of the normal control group and the disease state is significant.

The term “differentially expressed gene (DEG)” used herein refers to a gene in which a difference in an expression level significantly increases or significantly decreases in an experimental group compared to a control group when sequencing is performed on two or more samples. A method of selecting a DEG may vary according to the experimental design. A one-to-one comparison method between two groups, a time series analysis method over time after treatment in three or more groups, a method of identifying genes specifically expressed in several tissues, and the like may be used, but are not limited thereto.

The DEG may be a DEG already identified in a disease, but is not limited thereto. The DEG may be identified from a gene that is not associated with a disease. In the case where the DEG is identified in a disease, the DEG may be up-regulated or down-regulated compared to a normal state without a disease.

The DEG may refer to all genes that are differently expressed in a particular entity.

The DEG may be obtained from prostate cancer, but is not limited thereto. In addition, the DEG obtained from the prostate cancer may be one or more selected from ZNF750, ERG, ID4, HOXD13, L3MBTL4, ZNF154, and ZNF655, but is not limited thereto.

Alternatively, the DEG may be obtained from breast cancer, but is not limited thereto. The DEG obtained from the breast cancer may be one or more selected from CAND1, HIF1 A, PSME4, LOC107984583, and TOB1, but is not limited thereto.

The DEG may be a transcription factor of an early or late disease gene.

The term “read” used herein refers to base pair information regarding an amount of analysis generated from a DNA or cDNA fragment included in a sequencing library, and refers to a fragment of a sequence (base sequence) that is output data from sequencing.

The term “read number (RN)” used herein refers to a count that is a number of a sequence (base sequence) of read matching each gene, which is the output data from sequencing.

The particular index gene may be found particularly for each DEG.

In detail, the index gene may be selected from a candidate group in which values calculated when obtaining normalized standard deviation after dividing the count of a particular DEG in control samples by each gene count in the entire human gene pool are arranged in descending order.

The index gene may be obtained via Equation below.

$$D R N ( Dir - transformed R N ) = \frac{R N ( D E G )}{R N ( a human gene )}$$

$$Normalized S T D E V ( N S D ) = \frac{S T D E V ( D R N )}{average ( D R N )}$$

RN(DEG): read number of DEG
RN(a human gene): read number of each gene in entire human gene pool

The candidate group may be determined from the top 30 from among small normalized standard deviation values. The candidate group may be determined from the top 20 from among small normalized standard deviation values. The candidate group may be determined from the top 10 from among small normalized standard deviation values. The candidate group may be determined from the top five from among small normalized standard deviation values. Although a particular top rank does not determine the quality of an index gene, in the case where an index gene is present for a particular DEG, the best index gene may usually be determined within the top 10.

At least one index gene may be selected from the candidate group. At least two index genes may be selected from the candidate group. At least three index genes may be selected from the candidate group. At least four index genes may be selected from the candidate group. At least five index genes may be selected from the candidate group. At least six index genes may be selected from the candidate group. At least seven index genes may be selected from the candidate group. At least eight index genes may be selected from the candidate group. At least nine index genes may be selected from the candidate group. At least ten index genes may be selected from the candidate group.

In an embodiment, the index gene may be selected from a candidate exhibiting a much lower p-value or showing much improved calling accuracy than a DEG normalized by TPM. The calling accuracy (CA) may refer to the ability to accurately detect or call information related to a gene or transcript from RNA sequencing data. The calling accuracy may be calculated by identifying that a numerical value expected to increase or decrease in a particular DEG gene compared to a control group is called.

The index gene reacts similarly to a DEG due to various environmental factors.

The index gene may be found in prostate cancer or breast cancer, but is not limited thereto. The index gene found in the prostate cancer may be one or more selected from TMEM176A, TNFAIP8L1, LPCAT4, CDHR1, TRIM45, GNA14, and LOC107985770. The index gene found in the breast cancer may be one or more selected from KDM64, BIRC6, TAF13, CLEC12B, CD300E, and IRAG2.

In an embodiment, the method may further include a sequencing process to obtain data for selecting a DEG and an index gene.

The term “sequencing” used herein refers to reading and discovering DNA and RNA sequences, which are genetic information of a living organism. For example, a technology may be used, such as targeted sequencing, single-molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, thermal sequencing, duplex sequencing, cycle sequencing, single base extension sequencing, solid-phase sequencing, high-throughput sequencing, massively parallel signature sequencing, emulsification PCR, co-amplification-PCR at low denaturation temperatures (COLD-PCR), multiplex PCR, sequencing by reversible dye terminators, paired end sequencing, short-term sequencing, exonuclease sequencing, sequencing by ligation, short-read sequencing, single-molecule sequencing, sequencing by synthesis, real-time sequencing, reverse terminator sequencing, nanopore sequencing, 454 sequencing, Solexa genome analyzer sequencing, Illumina sequencing, SOLID sequencing, or MS-PET sequencing, but is not limited thereto.

Data obtained by the sequencing may be used after undergoing a preprocessing process by fastp, Trim Galore!, Trimmatic, Cutadapt, BBDuk (BBMap), Skewer, PRINSEQ, or Soapnuke, but is not limited thereto.

The data obtained by the sequencing may be used after being aligned by HISAT2, HISAT1, Bowtie2, spliced transcripts alignment to a reference (STAR), TopHat, Burrows-Wheeler Aligner (BWA), Salmon, Ensemble, or Sailfish, but is not limited thereto.

The data obtained by the sequencing may undergo a process of removing or minimizing noise through total normalized unique molecular identifiers (NormUMI), negative binomial regression (NBR), denoising of single-cell RNA-seq data via constrained artificial (DCA), marker genes with count adjustment (MAGIC), or sparse autoencoder for variational inference of gene expression regulation (SAVER), but is not limited thereto.

The “read number” may be influenced by a technical factor or an environmental factor.

The term “technical variation (TV)” or “technical factor” used herein refers to a factor that may occur during an experimental process, such as noise or bias due to RNA extraction, library production process, or the like.

The term “environmental variation (EV)” or “environmental factor” used herein refers to an external factor that is not inherited, such as sleep, nutrition, hydration, and other stresses.

The environmental variation may include mitochondrial transcription. The mitochondrial transcription may include a hyper-mitochondrial transcription in which mitochondrial transcription proceeds excessively.

In an embodiment, the method may further include an operation of providing RNA sequencing data.

“RNA sequencing (RNA-seq)” used herein refers to a process of deciphering the base sequence of the entire RNA expressed by analyzing a transcript by using NGS. The RNA-seq may measure a quantitative change in gene expression through a function of a gene, a mechanism of expression regulation, alternative splicing, and comparison between particular samples. The RNA-seq may be used for measurement of not only sequence information and expression levels, maintenance of directionality (sense or antisense), accurate analysis of 3’-untranslated region (UTR), accurate prediction of a genetic structure, discovery of a new gene, and discovery of a fusion gene, with respect to all types of RNA such as mRNA, long noncoding RNA (lncRNA), microRNA (miRNA), short interfering RNA (siRNA), and piwi-interacting RNA (piRNA).

The RNA-seq data may be obtained from a public database, a biological sample, or the like, but is not limited thereto.

As used herein, the “biological sample” may be one or more selected from the group consisting of a tissue, a cell, blood, serum, plasma, saliva, cerebrospinal fluid, and feces and urine, but is not limited thereto. A tissue obtained from a region in which a disease occurs or is predicted to occur may be used.

As used herein, the term “next-generation sequencing (NGS)” refers to a high-speed analysis method of the base sequence of the genome and is characterized by processing a large number of DNA fragments in parallel unlike existing base sequence analysis methods. Tens of millions of DNA fragments may be sequenced at a time. An NGS platform includes single molecule real-time sequencing, pyrosequencing, Sanger sequencing, and the like. To perform NGS, a sequencing library needs to be produced, and the sequencing library may be produced in various methods. The sequencing library may generally be divided into a method of using a glass slide or a method of using metal beads, according to a method of immobilizing DNA that is intended to be sequenced. Subsequently, DNA fragments, which ligate an oligomer attached to a support, and a complementary adaptor, are hybridized. Subsequently, PCR reaction is performed by using the oligomer attached to the support as a primer.

As used herein, the term “normalization” refers to a process of organizing a data set to reduce redundancy and improve data integrity, including adding adjustments to align adjusted values or fit a particular distribution. The normalization may eliminate systemic variations, such as experimental conditions or variability in machine parameters, and enable unbiased comparison across samples. In general, methods based on fragments per kilobase of transcripts per million mapped reads (FPKM), reads per kilobase of transcript per million mapped reads (RPKM), transcripts per million (TPM), trimmed mean of M-value (TMM), and housekeeping genes may be used.

As used herein, the term “DEG-index-ratio (Dir) transformation” may enable unbiased comparison across samples in a broad sense, such as normalization. However, unlike the normalization applied to all transcripts, the Dir transformation refers to a method of finding an index gene specific to only a particular DEG gene, removing mutations in the particular DEG gene, and performing unbiased comparison by using the same.

As used herein, the term “fragments per kilobase of transcripts per million mapped reads (FPKM)” refers to a method of calculating an expression value by using the number of fragments that are paired reads per transcript, and may be used for analysis of paired-end reads. In detail, the FPKM refers to a method of correcting and calculating the expression value by dividing the number of fragments by the entire length of a gene and then multiplying the same by 109.

As used herein, the term “reads per kilobase of transcript per million mapped reads (RPKM)” corrects the number of reads derived from a corresponding gene by the total number of derived reads and the length of the gene, and may correct deviations that may occur during comparison between samples and gene comparison within samples. In detail, the RPKM refers to a method of correcting the number of reads by dividing the number of reads by the entire length of a gene and then multiplying the same by 109. The RPKM is similar to the FPKM, but differs in that the RPKM is a method of using the number of reads instead of the number of fragments.

As used herein, the term “transcripts per million (TPM)” refers to a normalization method based on the total number of reads, and may represent a relative expression level that may be compared between samples.

The Dir transformation includes a two-dimensional Dir transformation and a three-dimensional Dir transformation, and may be obtained by the read number of a DEG and the read number of index gene(s). In detail, the Dir transformation may be calculated by dividing the read number of a particular DEG by the read number of an index gene, or by dividing the read number of the DEG by a value obtained by adding squared values of respective read numbers of two index genes and then calculating a square root.

The two-dimensional Dir transformation may be calculated via Equation below.

$$Two - dimensional Dir transformation = \frac{R N ( D E G )}{R N ( index )}$$

RN(DEG): read number of DEG
RN(index): read number of index gene

The three-dimensional Dir transformation may be calculated via Equation below.

$$T hree - dimensional Dir transformation = \frac{R N ( D E G )}{\sqrt{{( \frac{R N ( index 1 )}{a} )}^{2} + {( \frac{R N ( index 2 )}{b} )}^{2}}}$$

RN(DEG): read number of DEG
RN(index1): read number of first index gene
RN(index2): read number of second index gene
a and b: Relative contribution ratios of respective index genes

The term “relative contribution ratio” used herein refers to an index indicating a relative influence of a particular gene, or expression or characteristics of the corresponding gene within RNA-seq data on the overall change. The relative contribution ratio may be calculated by dividing an expression value or characteristic of a particular gene by the overall variability or characteristic variability of a corresponding data set, but is not limited thereto.

A simpler Dir transformation using the three-dimensional Dir transformation may be calculated via Equation below.

$$T hree - dimensional Dir transformation = \frac{R N ( D E G )}{\sqrt{{( R N ( index 1 ) )}^{2} + {( R N ( index 2 ) )}^{2}}}$$

RN(DEG): read number of DEG
RN(index1): read number of first index gene
RN(index2): read number of second index gene

The multidimensional Dir transformation may reduce an individual variation in data by removing an environmental factor.

In an embodiment, a multidimensional Dir transformation using the index gene may include an operation of additionally performing clustering to remove environmental variations.

The term “clustering” used herein refers to a process of grouping genes or samples having similar expression patterns. The clustering may be analyzing data by dividing the data into groups on the basis of a similarity between genes or between samples.

The clustering may be performed by using an algorithm such as hierarchical clustering, K-average clustering, DBSCAN, or t-SNE, but is not limited thereto.

The clustering may be performed by using an estimated DEG. The clustering may be performed by calculating a value of a correlation coefficient (r) by using the estimated DEG.

In an embodiment, the Dir transformation using the index gene may include an operation of calculating a correlation coefficient value of data used to remove a technical variation.

The Dir transformation using the index gene may be performed directly in a single RNA-seq experiment by inputting an absolute value (blood pressure or the like).

The Dir transformation using the index gene may define a normal range of a disease gene as a constantly narrow range and thus provide information enabling an early diagnosis of a disease.

The term “disease” used herein refers to a state in which the whole or part of the mind or body is primarily or continuously impaired and thus may not function normally. A disease may appear in chest, liver, thyroid, ears, musculoskeletal system, brain, eyes, legs, gallbladder, colon, lymph, head, neck, foot, abdomen, urinary system, spleen, genitalia, small intestine, hand, nervous system, kidney, cardiovascular system, shoulder, face, stomach, breast, uterus, mouth, spine, pancreas, or the like.

In an embodiment, the disease may be cancer, but is not limited thereto. The term “cancer” used herein refers to a physiological state in an animal generally having a characteristic of abnormal or uncontrolled cell growth. Cancer may be associated with, for example, metastasis, interference with normally functioning surrounding cells, release of cytokines or other secretion products at abnormal levels, inhibition or increase of inflammatory or immunological responses, neoplasia, premalignant, malignancy, surrounding or distant tissues or organs, e.g., lymph node invasion or the like.

The cancer may be solid cancer or blood cancer.

As used herein, the term “solid cancer” refers to cancer that has characteristics distinct from blood cancer and includes a lump formed by abnormal cell growth in several solid organs such bladder, breast, intestine, kidney, lung, liver, brain, esophagus, gallbladder, ovary, pancreas, stomach, cervix, thyroid, prostate, and skin.

The solid cancer may be melanoma, brain tumor, benign astrocytoma, malignant astrocytoma, pituitary adenoma, meningioma, brain lymphoma, oligodendroglioma, ependymoma, brainstem tumor, head and neck tumor, laryngeal cancer, oropharyngeal cancer, nasal cavity/paranasal sinus cancer, nasopharyngeal cancer, salivary gland cancer, hypopharyngeal cancer, thyroid cancer, oral cancer, thoracic tumor, small cell lung cancer, non-small cell lung cancer, thymic cancer, mediastinal tumor, esophageal cancer, breast cancer, male breast cancer, abdominal tumor, stomach cancer, liver cancer, gallbladder cancer, biliary tract cancer, pancreatic cancer, small intestine cancer, colon cancer, rectal cancer, anal cancer, bladder cancer, kidney cancer, male genital tumor, penile cancer, prostate cancer, female genital tumor, cervical cancer, endometrial cancer, ovarian cancer, uterine sarcoma, vaginal cancer, female external genital cancer, female urethral cancer, bone tumor, duodenal cancer, fibrosarcoma, or skin cancer, but is not limited thereto.

As used herein, the term “blood cancer” refers to cancer that occurs in the components of blood and refers to a malignant tumor that occurs in blood, hematopoietic organs, lymph nodes, lymphatic organs, or the like.

The blood cancer may be acute myeloid leukemia, acute lymphocytic leukemia, chronic myeloid leukemia, chronic lymphocytic leukemia, acute monocytic leukemia, multiple myeloma, Hodgkin lymphoma, or non-Hodgkin lymphoma, but is not limited thereto.

The disease may be prostate cancer or breast cancer.

In an embodiment, an entity in which the disease occurs may be a mammal, but is not limited thereto.

The mammal may be a human, a cow, a horse, a pig, a dog, a sheep, a goat, or a cat, but is not limited thereto.

An operation of determining whether or not a difference between DEG expressions of a normal control group and a disease state is significant may include an operation of determining as the disease state or selecting as a DEG that is significant for determining the disease state in the case where the difference between the normal control group and the DEG is significant, or determining not to be the disease state or selecting as a DEG that is insignificant for determining the disease state in the case where the difference is insignificant.

Another aspect provides a method of selecting a gene that is significant for a disease state including: selecting at least one particular index gene from among all genes by using a differentially expressed gene (DEG) differentially expressed in a disease state compared to a normal control group; performing a DEG-index-ratio (Dir) transformation using the DEG and an index gene; and determining whether or not a difference between DEG expressions of the normal control group and the disease state is significant.

A gene significant for the disease state may be used as a biomarker used to diagnose a disease.

The gene may be one or more selected from DEGs in which a difference between expressions a normal control group and a disease state is significant.

The term “biomarker” used herein refers to a substance that may be used to make a diagnosis by distinguishing between a normal entity and an entity with a disease. The biomarker may be one or more selected from organic biomolecules such as polypeptides, proteins or nucleic acids, genes, lipids, glycolipids, glycoproteins, or sugars that are shown to increase or decrease in an entity having a disease.

The biomarker may be a gene that changes in an entity having a disease, but is not limited thereto.

Another aspect provides a method of diagnosing a disease state including: selecting at least one particular index gene from among all genes by using a differentially expressed gene (DEG) that is differentially expressed in a disease state compared to a normal control group; performing a DEG-index-ratio (Dir) transformation using the DEG and the index gene; and determining whether or not a difference between DEG expressions of the normal control group and the disease state is significant.

Another aspect provides a method of treating a disease state including: selecting at least one particular index gene from among all genes by using a differentially expressed gene (DEG) that is differentially expressed in a disease state compared to a normal control group; performing a DEG-index-ratio (Dir) transformation using the DEG and the index gene; and determining whether or not a difference between DEG expressions of the normal control group and the disease state is significant.

Another aspect provides a recording medium having recorded thereon a method for causing a computer to execute the method described above.

The recording medium may be implemented as an application (or program) and readable by a terminal device (or a computer). In detail, the recording medium may include any type of recording device or medium that stores data readable by a computing system.

### Advantageous Effects of Invention

According to a method of performing a Dir transformation by finding a particular index gene for a DEG, according to an aspect, not only individual environmental factors may be reduced in RNA-seq data, but also a disease may be diagnosed in a single RNA-seq experiment by inputting an absolute value that distinguishes between a normal state and a disease state, and thus, the method may be useful to provide information for an early diagnosis of a disease.

### BRIEF DESCRIPTION OF DRAWINGS

FIG. 1 is a schematic view illustrating a method of providing information for diagnosing a disease, according to an embodiment.

FIG. 2 illustrates eight GstD genes located at the genetic locus of chr3R in Drosophila RNA-seq data.

FIG. 3 illustrates five GstD genes GstD2, GstD4, GstD5, GstD6, and GstD7 identified as inducible DEGs from among eight GstD genes of Drosophila.

FIG. 4 illustrates three GstD genes GstD3, GstD8, and GstD11 identified as non-DEGs from among eight GstD genes of Drosophila.

FIG. 5 illustrates a similarity between expression patterns of two DEGs GstD4 and GstD6 and a non-DEG GstD8 in RNA-seq of repeated experiments treated with ML DmT1 to DmT5.

FIG. 6 illustrates a dispersion distribution of gene expression patterns in control RNA-seq DmC1 to DmC5.

FIG. 7 illustrates GstD4 and GstD6 on which a TPM or Dir transformation is performed in an RNA-seq experiment treated with ML.

FIG. 8 illustrates GstD4 and GstD6 on which a TPM or Dir transformation is performed in nine times-repeated RNA-seq experiments treated with ML.

FIG. 9 illustrates a schematic view of a Dir transformation for reducing an environmental variation.

FIG. 10 illustrates the result of identifying a DEG in human prostate cancer RNA-seq data.

FIG. 11 illustrates the result of ZNF655 on which a TPM or Dir transformation is performed.

FIG. 12 illustrates the result of HOXD13 on which a TPM or Dir transformation is performed.

FIG. 13 illustrates the result of ID4 on which a TPM or Dir transformation is performed.

FIG. 14 illustrates gene expression patterns of ZNF655 and five types of index genes in a normal tissue sample, and illustrates TPM nTPM normalized with average TPM of the normal tissue sample.

FIG. 15 illustrates gene expression patterns of ZNF655 and five types of index genes in a tumor sample, and illustrates TPM nTPM normalized with average TPM of the tumor sample.

FIG. 16 illustrates a portion of similar expression (PSE) between ZNF655 and five index genes.

FIG. 17 illustrates that normalized TPM nTPM of ZNF655 and five types of index genes is generally present within an nTPM value of the entire transcript.

FIG. 18 illustrates elevated TPM levels of 16 mitochondrial (MT) transcripts in 10NRNA-seq.

FIG. 19 illustrates a ratio of RNs of 16 mitochondrial transcripts in a total of 12,487 genes.

FIG. 20 illustrates a distribution of modified nTPM in 12,372 genes from which 16 mitochondrial transcripts are excluded.

FIG. 21 illustrates that removing hyper MT variation improves calling accuracy and decreases a p-value of a ZNF655 modified TPM.

FIG. 22 illustrates a TPM or Dir transformation RN(Cd68)/RN(Adgre4) in a tumor-induced mouse.

FIG. 23 illustrates a TPM or Dir transformation RN(Msr1)/RN(Abca9) in a tumor-induced mouse.

FIG. 24 illustrates a TPM or Dir transformation RN(Gtf2i)/RN(Tap1) in a tumor-induced mouse.

FIG. 25 illustrates a TPM or Dir transformation RN(Ctbp1)/RN(Prelid1) in a tumor-induced mouse.

FIG. 26 illustrates a TPM or Dir transformation RN(HIF1A)/RN(KDM6A) of human monocyte RNA-seq data of a breast cancer patient.

FIG. 27 illustrates a TPM or Dir transformation RN(PSME4)/RN(BIRC6) of human monocyte RNA-seq data of a breast cancer patient.

FIG. 28 illustrates a TPM or Dir transformation RN(TOB1)/RN(TAF13) of human monocyte RNA-seq data of a breast cancer patient.

FIG. 29 illustrates a TPM or Dir transformation RN(LOC107984583)/RN(CLEC12B) of human monocyte RNA-seq data of a breast cancer patient.

FIG. 30 illustrates three examples of prostate cancer tissue RNA-seq to compare a Dir transformation with SMART noise removal.

FIG. 31 illustrates the result of performing a Dir transformation by using ID4 that is a DEG and GNA14 that is an index gene.

FIG. 32 illustrates the result of performing a Dir transformation by using ID4 that is a DEG and LOC107985770 that is an index gene.

FIG. 33 illustrates the results of performing a two-dimensional Dir transformation and a three-dimensional Dir transformation by using ID4 that is a DEG and GNA14 and LOC107985770GNA14 that are index genes.

### MODE FOR THE INVENTION

Hereinafter, example embodiments are presented to help understand the present disclosure. However, the following embodiments are only provided to more easily understand the present disclosure, and the present disclosure is not limited by the following embodiments. The embodiments may be modified in various ways, and the embodiments are not limited to embodiments provided below and may be implemented in various forms.

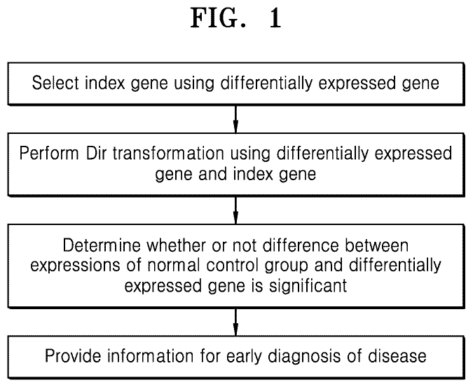

FIG. 1 is a schematic view illustrating a method of providing information for diagnosing a disease, according to an embodiment.

Referring to FIG. 1, a method of providing information for diagnosing a disease, according to an embodiment, may include the operations of: selecting at least one particular index gene from among all genes by using a differentially expressed gene (DEG) that is differentially expressed in a disease state compared to a normal control group; and performing a DEG-index-ratio (Dir) transformation using the DEG and the index gene to determine whether or not a difference between DEG expressions of the normal control group and the disease state is significant.

### Reference Example 1. Processing of RNA-seq data

After acquiring a sequence from RNA-seq data or data acquired from an NCBI SRA database, resulting sample FASTQ files from all batches run through the same processing pipeline by using a Galaxy server (usegalaxy.org), except for using different genomes and corresponding annotation files. Single-end RNA-seq data was preprocessed by fastp and then mapped to each genome by using HISAT2. A mapped BAM file was applied to featureCounts to acquire an RN(an NCBI reference gene) of each transcript.

### Example 1. Identification of Proposal and Possibility of Dir Transformation

### 1.1. Proposal for Dir Transformation

To perform normalization that may exclude environmental factors, two-dimensional normalization using an index gene was proposed. In detail, a Dir transformation was expressed as a value calculated by dividing the read number of a DEG by the read number of an index gene, and was calculated through Equation below.

$$Two - dimensional Dir transformation = \frac{R N ( D E G )}{R N ( index )}$$

RN(DEG): read number of DEG
RN(index): read number of index gene

### 1.2. Identification of Dir Transformation Hypothesis Using DEG

To identify a two-dimensional normalization hypothesis proposed in Example 1.1 above, a defense mechanism of Drosophila melanogaster against methyl lucidone (ML), which is a plant diterpene, was investigated in an RNA-seq experiment. In detail, 20 male adults and 20 female adults of Drosophila melanogaster were placed in individual vials, and then 3g of artificial feed mixed with 0.5% ml (w/v) or 0.5% ethanol (w/v) was added to each vial. Two days after spawning, Drosophila was taken out of each vial and eggs laid were developed and used. Second-instar larvae were collected from each vial 4 to 5 days after spawning. Subsequently, the total RNA of the collected larvae was separated by using an RNeasy separation kit (Qiagen, Hilden) according to instructions of a manufacturer. Data was obtained by performing Illumina RNA sequencing by using the separated RNA. Transcripts of a control group and an ML-treated drosophila were compared by using the obtained data, and five repeated experiments were performed. In the process, a total of 73 million to 132 million reads were generated, and from among the same, 50 million to 95 million reads were assigned to annotated genes (Drosophila reference genome dm6). Subsequently, RNA-seq data was processed through the method of Reference Example 1.

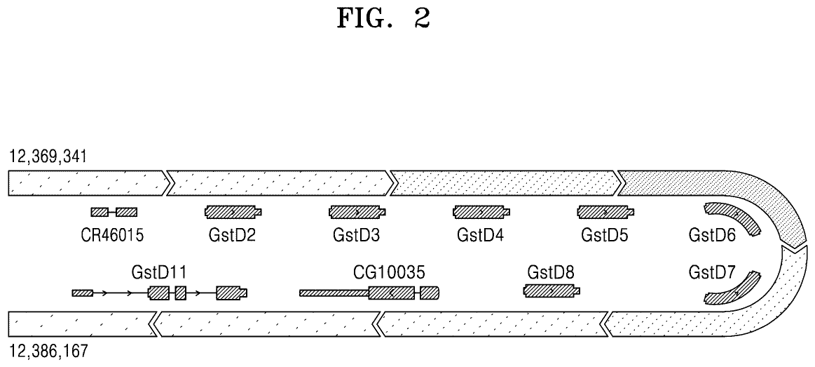

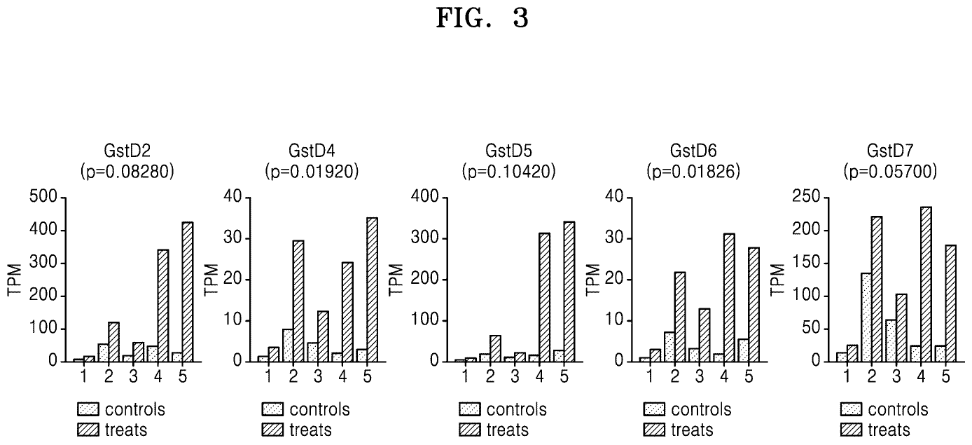

Due to a great difference between gene expression values (TPM) in five repeated independent experiments, a DEG was manually identified by using an integrated genomics viewer (IGV). As illustrated in FIG. 2, eight of 11 GstD genes were identified to be at genomic locations chr3R: 12,369,341 to 12,386, 167. While manually investigating a DEG induced by ML in drosophila, as illustrated in FIG. 3, five genes GstD2, GstD4, GstD5, GstD6, and GstD7 from among the eight genes were identified to be inducible DEGs in all of five repeated experiments.

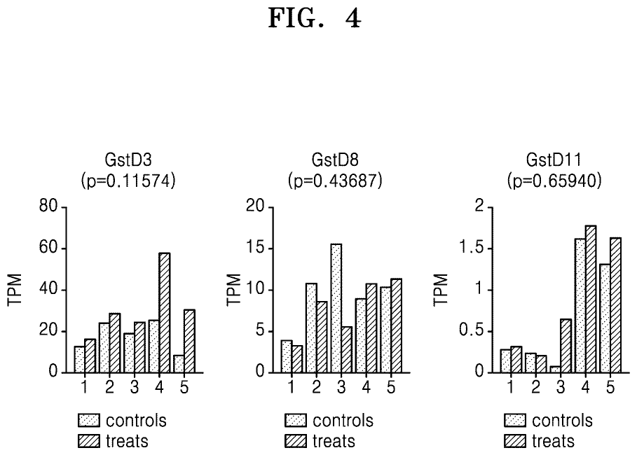

However, a great difference occurs between repeated experiments, and thus, p-values of two sample t-tests exceed 0.01 in five GstD genes. As illustrated in FIG. 4, the remaining three genes GstD3, GstD8, and GstD11 were identified to be non-DEGs.

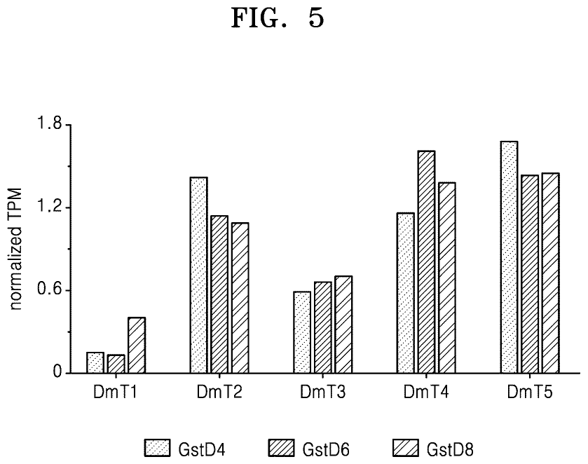

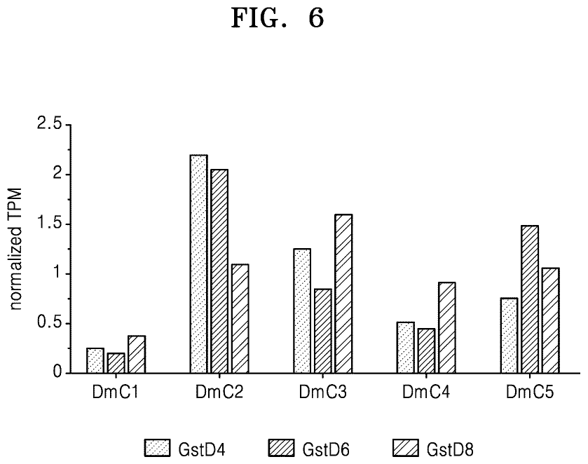

From among the same, three GstD genes (two DEGs GstD4 and GstD6) and one non-DEG GstD8) were used to identify a similarity between expression patterns expressed by transcripts per million (TPM). As illustrated in FIG. 5, a similarity among expression patterns of three GstD genes was identified in a sample DmT treated by ML. In contrast, as illustrated in FIG. 6, greatly various expression patterns of a GstD gene appear in a control sample DmC.

Therefore, in the case where different environmental conditions equally affect expressions of three GstD genes, an environmental influence may reduce an influence on a DEG expression through a Dir transformation that divides the RN of a DEG GstD4 or GstD6 by the RN of a non-DEG GstD8.

### 1.3. Comparison of Existing Normalization and Dir Transformation Using DEG

Accuracy of a Dir transformation was to be identified by comparing the Dir transformation with TPM that was an existing normalization method. In detail, DEGs GstD4 and GstD6 used in Example 1.2 were used, and a Dir transformation for normalizing to TPM or dividing each RN by the RN of the non-DEG GstD8 was performed. Calling accuracy (CA) does not change with an existing expression method for RNA-seq data, such as existing TPM, reads per kilobase of exon per million reads mapped (RPKM), or fragments per kilobase million (FPKM). Therefore, RNA-seq data was represented by using TPM for comparison with a Dir transformation.

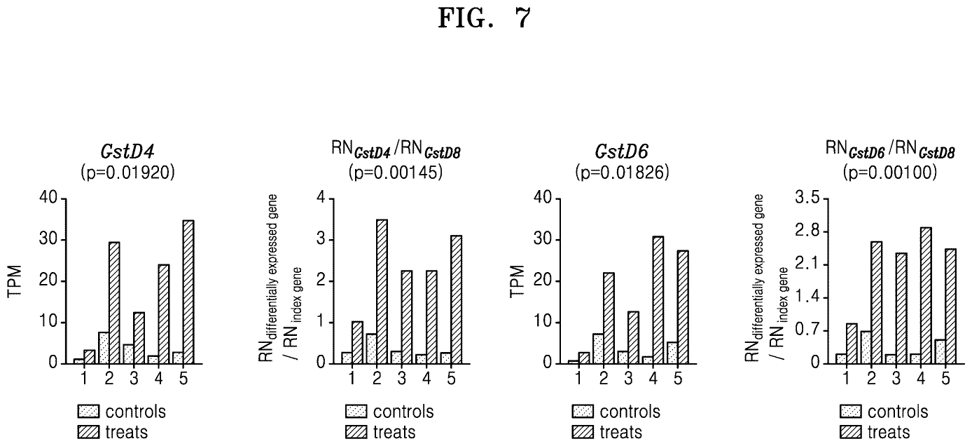

As illustrated in FIGS. 5 and 6, when comparing numerical values of ML treatment groups and control groups for two DEGs GstD4 and GstD6, calling accuracy was identified to be 4/5. The above example indicates that, from among five repeated data of GstD4 or GstD6, TPM values of four ML treatment groups were greater than the highest value of a control sample. In contrast, as illustrated in FIG. 7, a Dir transformation for an RN(GstD4)/RN(GstD8) or an RN(GstD6)/RN(GstD8) showed perfect calling accuracy. Accordingly, all ratios of an ML treatment group were distinguished from a control sample. In addition, as the result of estimating the quality of calling accuracy by using a p-value of a t-test between an ML treatment group and a control group, a Dir transformation lowered the p-value by improving the quality of calling accuracy, and the quality was improved by about 10 times compared to TPM.

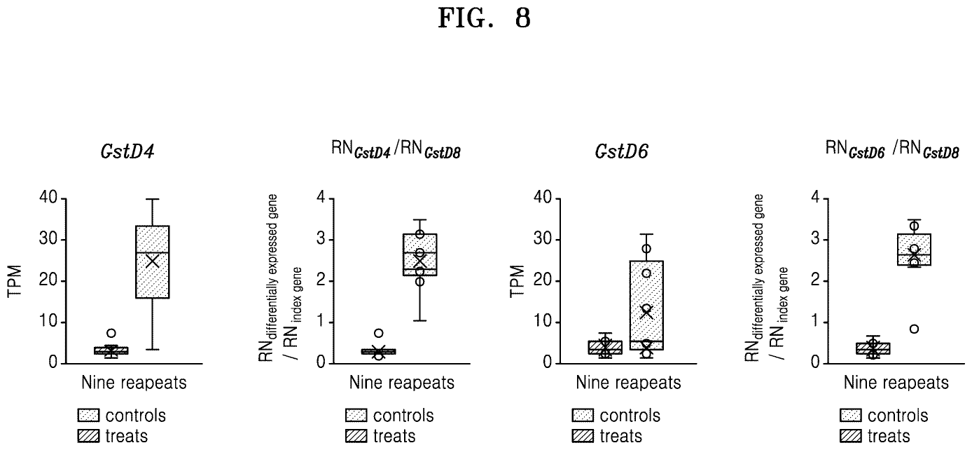

Four sets of RNA samples were additionally sequenced to further verify a hypothesis about the Dir transformation. As illustrated in FIG. 8, calling accuracy for a Dir transformation was perfectly identified to be 9/9 in 18 RNA-seq data sets through nine repeated experiments, and a p-value for the Dir transformation was identified to be 2.67E-07 for an RN(GstD4)/RN(GstD8) and 2.98E-07 for RN(GstD6)/RN(GstD8).

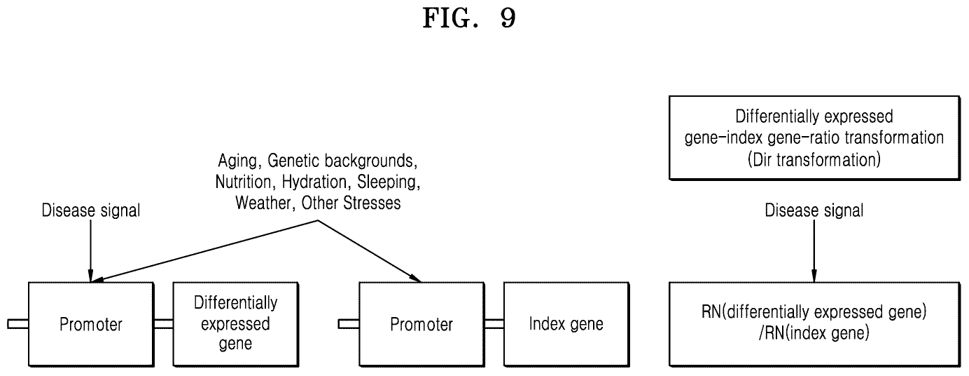

On the basis of the above results, as illustrated in FIG. 9, a hypothesis was established about that an expression of a disease-related DEG is affected by an environmental variation as well as by a disease signal and a Dir transformation for an RN(DEG)/RN(Index) reduces RNA-seq data variation caused by the environmental variation. It was assumed that, in the case where an index gene has a similar expression pattern to a DEG during repeated experiments for RNA-seq, expressions of the DEG and a particular index gene responds similarly to environmental variations. Both the RN of a DEG and a particular index gene were obtained by single performance of the same RNA-seq experiment, and the same indicates that the environmental variation was the same for all genes. An expression of an index gene does not respond to a disease signal, and thus, an environmental variation may be reduced and a DEG expression may be normalized, by calculating RN(DEG)/RN(Index) according to the disease signal.

### Example 2. Application of Dir Transformation

### 2.1. Index Gene Selection Method

To select a reliable index gene for a particular DEG, the following method was performed. In detail, an average read number (RN) was set to a value greater than or equal to 50. Subsequently, for a normal or control sample, the RN of a DEG was divided by the RN of all genes, and then normalized standard deviation (NSD) was obtained. The normalized standard deviation was calculated by using Equation below.

$$D R N ( Dir - transformed R N ) = \frac{R N ( D E G )}{R N ( a human gene )}$$

$$Normalized S T D E V ( N S D ) = \frac{S T D E V ( D R N )}{average ( D R N )}$$

RN(DEG): read number of DEG
RN(a human gene): read number of each gene in entire human gene pool

Values of the NSD calculated by Equation above were arranged in descending order, and from among the same, the lowest ten genes were considered index candidate genes. Subsequently, a Dir transformation selects an index gene from candidates that exhibit much lower p-values and show much improved calling accuracy than a DEG normalized by TPM. Index genes used for two-dimensional normalization were obtained from the same RNA-seq experiment.

### 2.2. Identification of Dir Transformation in Prostate Cancer

Prostate cancer data was used to prove a selection method for the index gene and a Dir transformation by an RN(DEG)/RN(Index). In detail, a total of 28 RNA-seq datasets including 14 primary prostate cancer samples and 14 normal samples paired with the prostate cancer samples were retrieved from an NCBI SRA database. The datasets were processed by the method of Reference Example 1 by using a human reference genome (GRCh38).

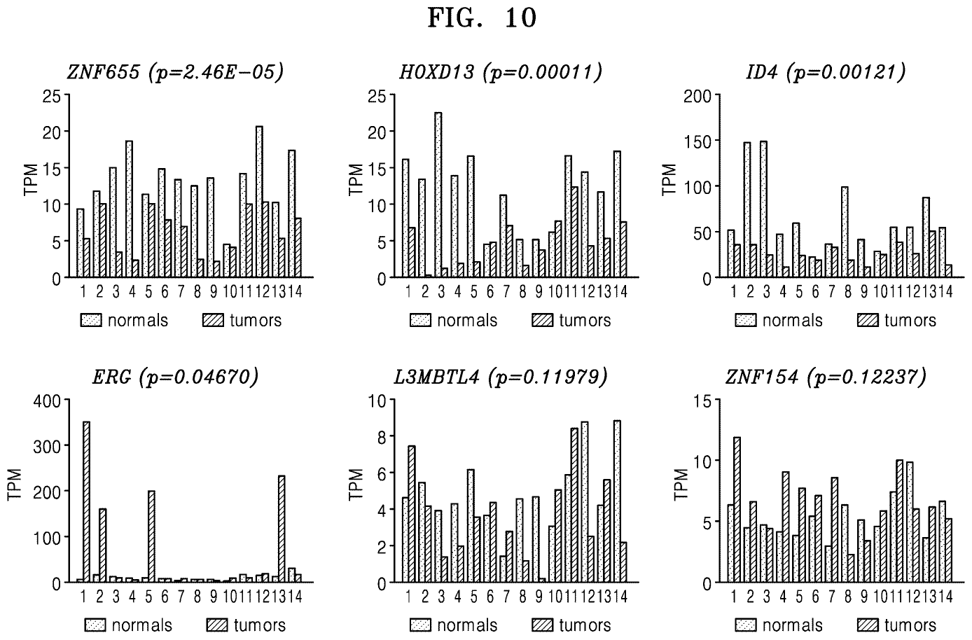

To determine a DEG in prostate cancer, six transcription factors ZNF655, HOXD13, ID4, ERG, L3MBTL4, and ZNF154, which were mainly identified in prostate cancer, were considered. As illustrated in FIG. 10, expressions of six transcription factor genes were investigated in each of 14 normal and prostate cancer samples, and from among the same, ZNF655, HOXD13, and ID4 having p-values identified to be less than 0.01, were identified as DEGs that were significantly down-regulated.

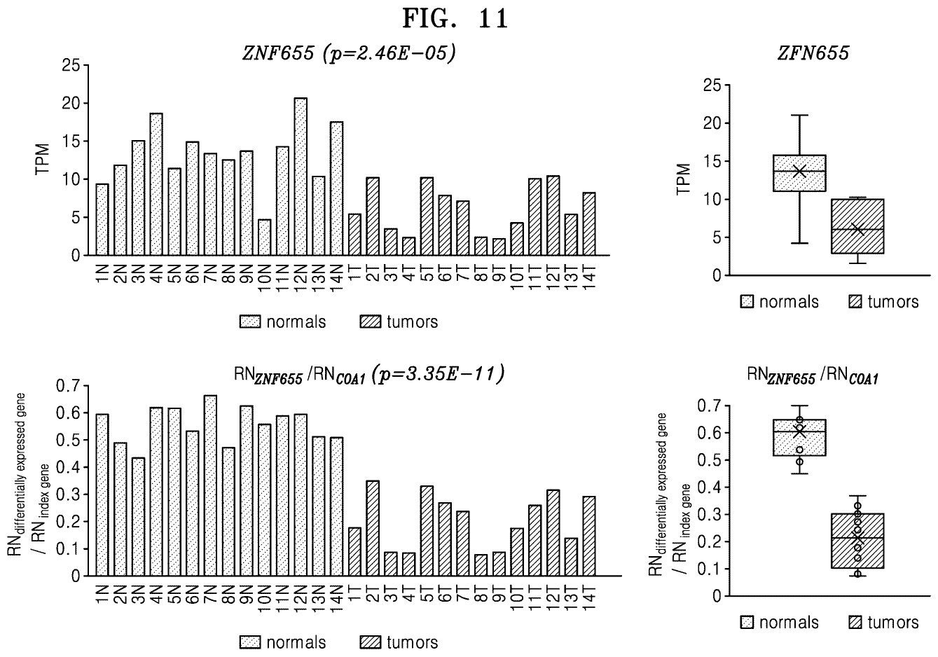

Subsequently, to determine an index gene, a gene, which has low NSD and in which Dir transformation showing lower p-value and calling accuracy than DEG TPM was performed, was selected. In detail, to determine a particular index gene for ZNF655, 12,487 richly expressed genes having an average RN exceeding 100 were selected. Subsequently, after dividing the RN of ZNF655 by the RN of other 12,486 genes, 10 genes having the lowest NSD score of each RN(ZNF655)/RN(other genes) in 14 normal samples were considered as index candidate genes. Subsequently, five index genes TAF11, UBE2A, COA1, PPP6R3, and FBXO42 having at least 100-fold lower p-values and perfect calling accuracy were selected from among 10 candidates by using a Dir transformation in ZNF655 analysis. From among the five index genes, COA1 showed the lowest p-value for the Dir transformation. As illustrated in FIG. 11, a Dir transformation of RN(ZNF655)/RN(COA1) showed a 6.93E+05-fold increase in a p-value and perfect calling accuracy. Also, a box plot indicated that the Dir transformation of RN(ZNF655)/RN(COA1) successfully normalizes ZNF655 RN to perfectly distinguish between a normal tissue and a tumor tissue.

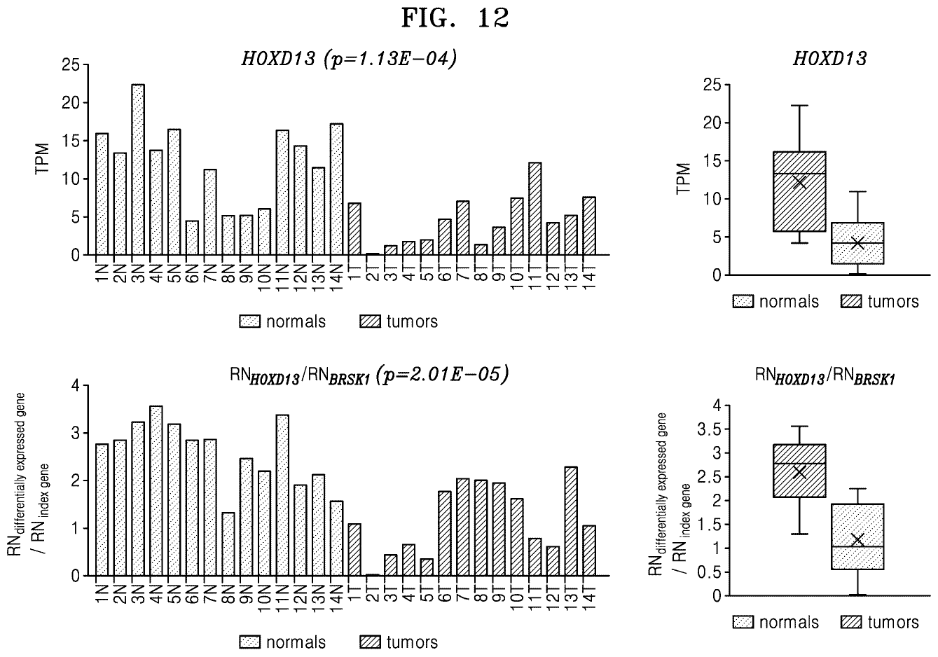

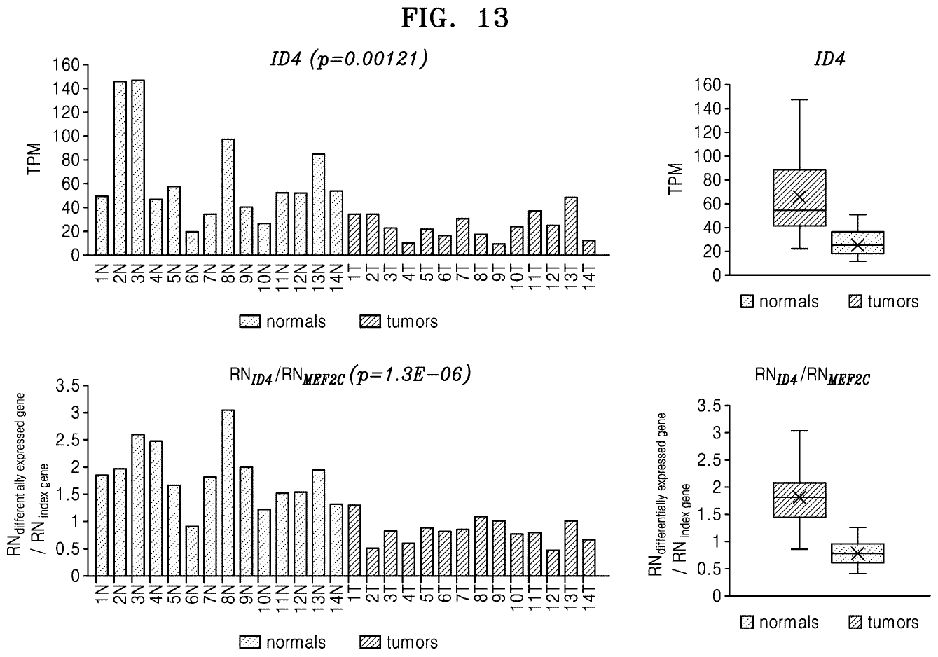

The Dir transformation was performed even on the other DEGs HOXD13 and ID4 by using the above method. As illustrated in FIGS. 12 and 13, for the two types of Dir transformations, RN(HOXD13)/RN(BRSK1) and RN(ID4)/RN (MEF2C) partially remove environmental variations and thus improve calling accuracy.

### 2.3. Identification of Natural Presence of Index Gene

To identify whether or not an index gene was randomly generated, an expression pattern of the index gene was identified in a normal sample and a prostate cancer sample.

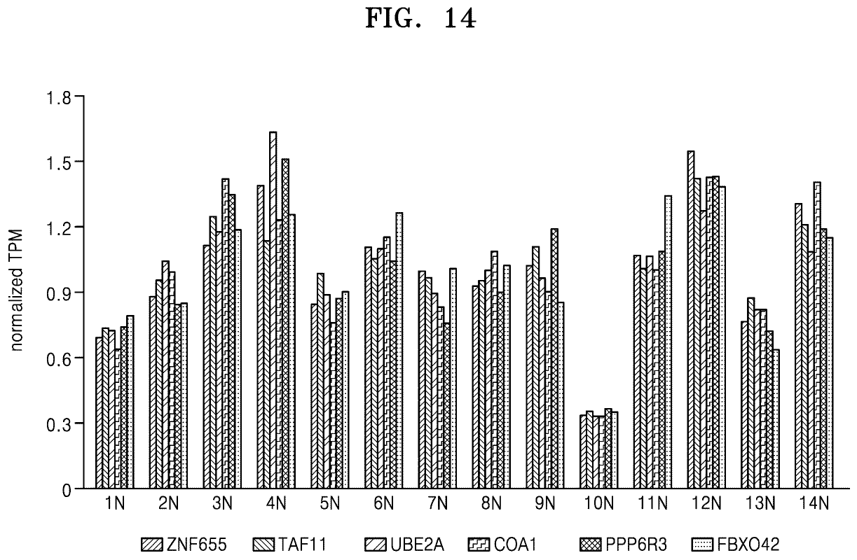

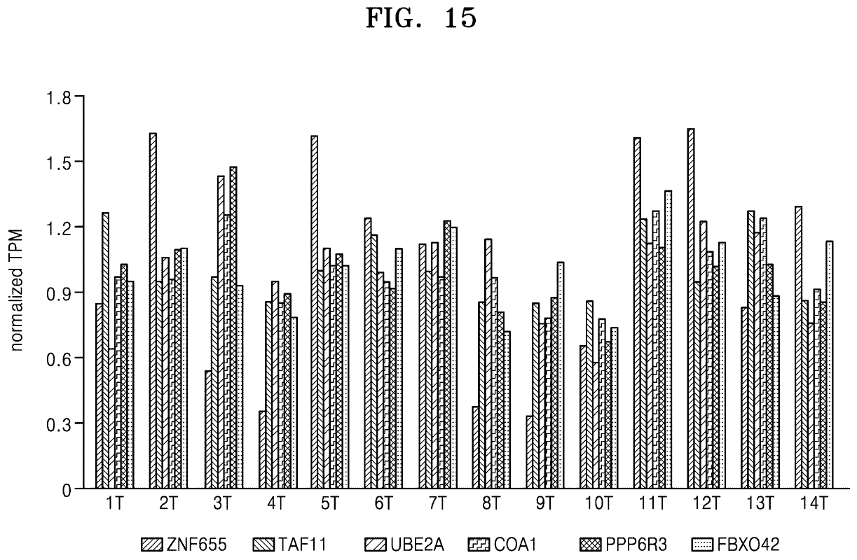

As illustrated in FIG. 14, similar expression patterns in ZNF655 and five index genes were identified in 14 normal samples. In addition, as illustrated in FIG. 15, similar expression patterns in ZNF655 and five index genes were identified even in 14 tumor samples.

A Dir transformation was forcibly performed by other 12,486 genes, and thus, a particular index gene for a DEG in prostate cancer may also be randomly generated while executing a protocol. However, a portion of similar expression (PSE) between ZNF655 and five index genes thereof showed that a probability of random generation of a particular index gene for ZNF655 was greatly low. The PSE was calculated as the number of portions that were between the lowest or highest nTPM value ranges of ZNF655 and five index genes thereof from among normalized TPM (nTPM) values of 12,487 genes. For example, the lowest nTPM and the highest nTPM at 1N were 0.640 for COA1 and 0.788 for FBXO42, respectively, and account for 25% of a total of 12,487 genes.

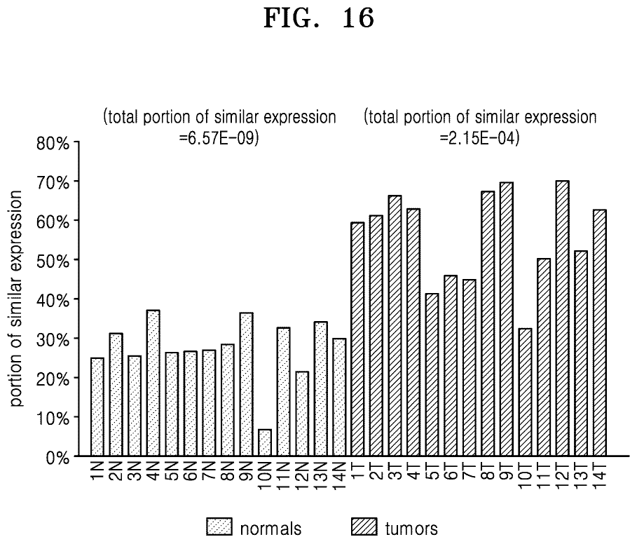

As illustrated in FIG. 16, in the case of 14 normal samples, a PSE was 8 to 37% and an average of 28%. In contrast, in the case of 14 tumor samples, a PSE averages 56% and was identified between 36% and 70%. Subsequently, a total PSE for 14 normal or tumor samples was calculated by multiplying respective PSEs in 14 samples. The total PSE for 14 normal samples was 6.57E-09 and was identified to be a greatly low value. Considering that simple 12,487 genes were tested, results suggest that a particular index gene for ZNF655 was naturally present and was not randomly generated. In contrast, the total PSE (2.15E-04) for 14 tumor samples was identified to be much higher than the total PSE for the 14 normal samples.

The above results show that an index gene that reacts similarly to a DEG was selected according to environmental factors.

### Example 3. Identification of Influence of Hyper-mitochondrial Transcription (Hyper-MT)

A Human blood transcript from a large population cohort showed that signs of several diseases were influenced not only by diseases but also by aging or health. In addition, age and presence of non-blood cancer similarly affects a blood transcript, and a high blood concentration of hemoglobin mRNA affects calling and quantification of non-hgbRNA. All the results show that blood RNA-seq data was greatly affected by environmental variations. Therefore, an attempt was made to identify whether or not a variation of hyper-mitochondria transcription (hyper-MT) also affects as one of the environmental variations. In detail, prostate cancer RNA-seq data used in Example 2 was used, and identified by TPM normalization by using DEG ZNF655 and five index genes.

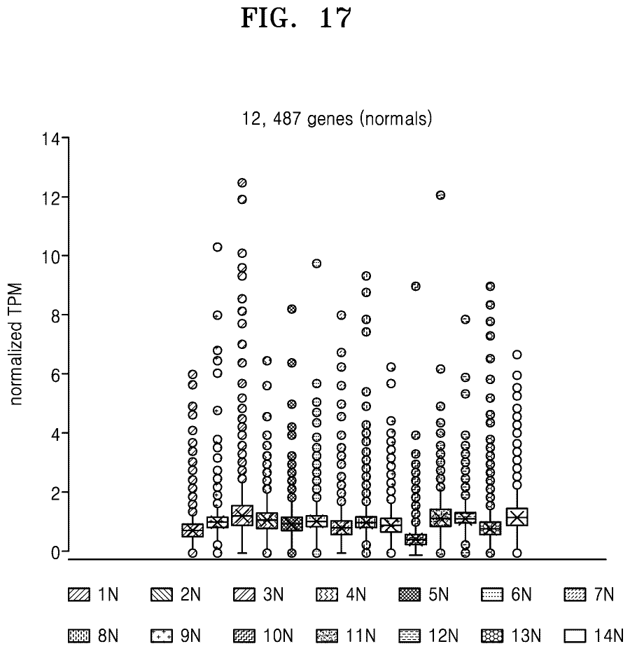

As illustrated in FIG. 15, the nTPM (normalization of TPM) of ZNF655 and five index genes was identified to be low at 10N, and hyper-MT was identified to affect an expression of ZNF655. The normalization of nTPM was performed as an average of the TPMs of respective transcripts in 14 normal samples, and accordingly, TPMs of a transcript at 10N was expected to be lower than in other 13 normal samples. As expected (FIG. 17), the distribution pattern of nTPM in all transcripts showed that low TPM at 10N was present in most of 12,487 genes. Accordingly, some transcripts of 10N RNA-seq samples were extremely abundant, and thus, other genes at 10N may be allowed to have lower TPM to lower nTPM.

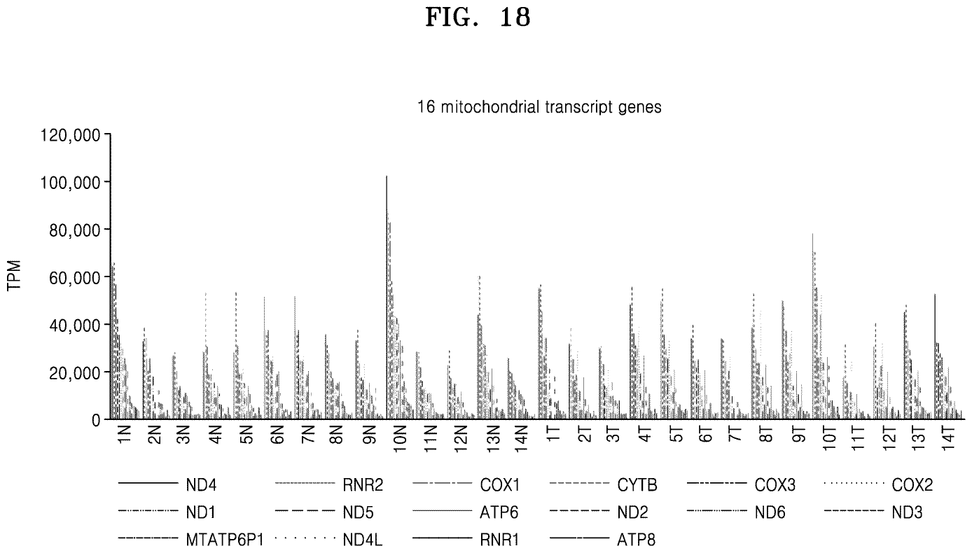

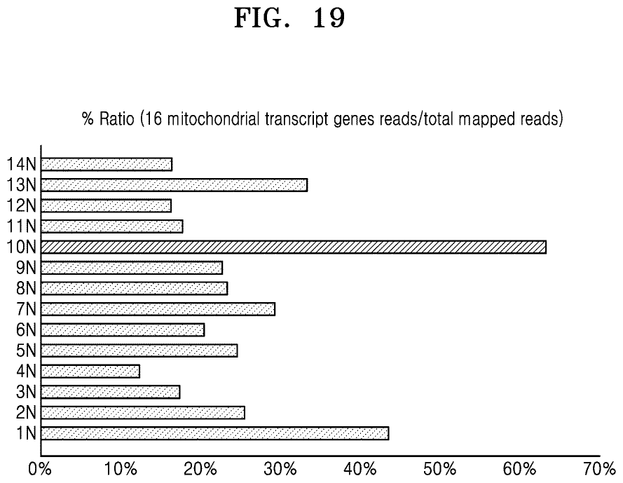

Therefore, TPM values of 12,487 genes were arranged from the highest value and identified. As illustrated in FIG. 18, the top 16 transcripts having the highest TPM at 10N were identified to be all derived from mitochondrial (MT) genes. In particular, TPM of 16 MT transcripts at 10N was identified to be 2.62 times higher than an average of TPM in other 13 normal samples. As a result of dividing reads of the 16 MT transcripts by reads of the entire map to identify a ratio, as illustrated in FIG. 19, the RN of 16 MT transcripts accounted for 63% of the RN of a total of 12,487 genes at 10N. In contrast, in other 13 normal samples, the ratio was identified to be between 13% and 44%.

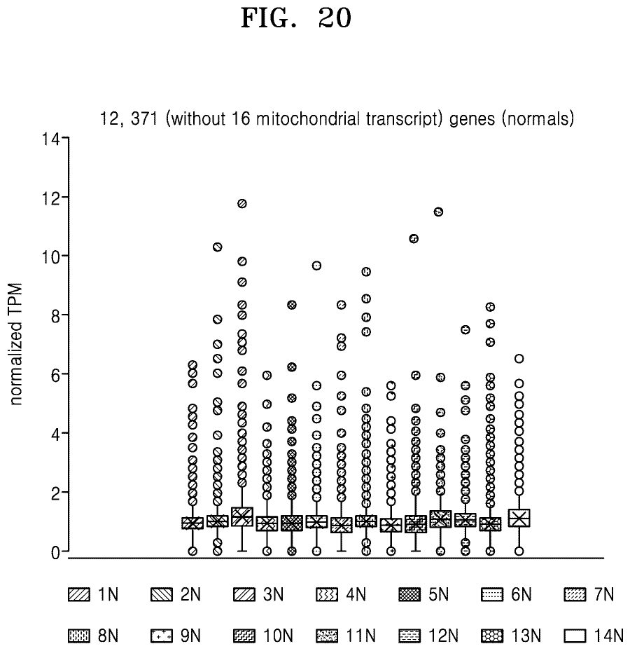

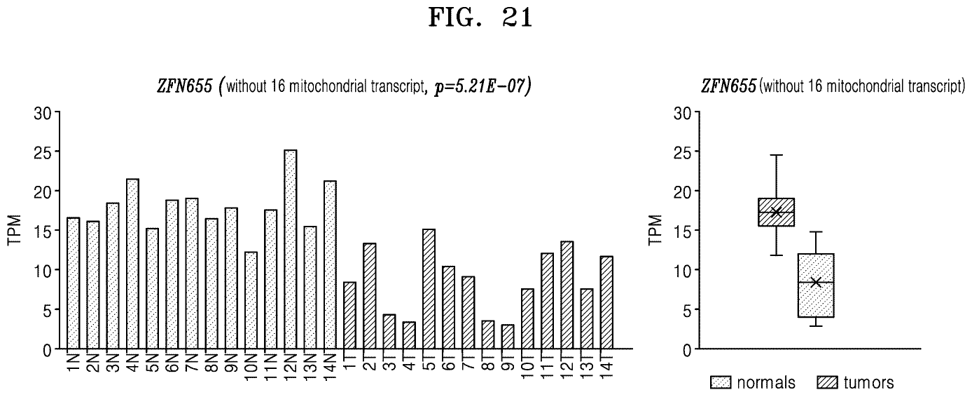

Subsequently, nTPM was modified, and calculated for 12,471 genes excluding the 16 MT transcripts. As illustrated in FIG. 20, when a distribution of modified nTPM was tested in 12,371 genes, a variation of the TPM shown in FIG. 17 was greatly reduced. Accordingly, the variation of the TPM arises from an MT transcript (hyper-MT variation) in which environmental variations were abnormally abundant. In addition, as illustrated in FIG. 21, as a result of removing hyper variations by using data excluding 16 MT transcripts, calling accuracy improves and a p-value decreases.

As identified from the above results, a Dir transformation eliminated not only hyper-MT variations but also other unknown environmental variations, and thus, as in the box plot shown in FIG. 11, a difference between gene expression of a normal state and a disease appears clearly.

### Example 4. Dir Transformation in Tumor-Induced Mouse

### 4.1. Method of Producing Tumor-induced Mouse and Sequencing Blood RNA Data

To determine whether or not a tumor-induced mouse may be used to detect a solid tumor even in whole-blood RNA-seq data through a Dir transformation, an experiment was performed by using the tumor-induced mouse. In detail, a murine colon adenocarcinoma (MC-38) cell line used to induce a tumor was purchased from Kerafast (Cat #: ENH204-FP, Boston, MA, USA) and used. The MC-38 was cultured in a medium including 10% fetal bovine serum (Thermo Fisher Scientific Inc.), 0.1 mM nonessential amino acid (Thermo Fisher Scientific Inc.), 100 U/mL penicillin, and 100 μg/mL streptomycin (Roche). A six-week-old female C57BL/6N mouse used in an experiment was purchased from Koatech (Pyeongtaek, Gyeonggi-do, South Korea) and reared and used under a particular pathogen-free condition. The six-week-old female C57BL/6N mouse was reared at 21±2° C. under a 12-hour light/dark period, and was adapted to a local environment for 1 week prior to use. All animal experiments were performed with the approval of Institutional Animal Care and Use Committee of the Korea Research Institute of Bioscience and Biotechnology (IACUC approved #: KRIBB-AEC-22256).

To induce a tumor, the cultured MC-38 cell was collected and suspended in a serum-free medium. A serum-free medium (200 μL) or cell suspension (1×10⁶ cells/mice) was injected subcutaneously into each of 10 C57BL/6 mice. Three weeks after the subcutaneous injection, blood was obtained into a heparinized blood collection tube by a retroorbital puncture method, and mixed with an RNAlater solution (Thermo Fisher Scientific) for later use. RNA was extracted from a mouse whole-blood sample stored in RNAlater at −70° C. by using the Mouse RiboPure Blood RNA Isolation Kit (Thermo Fisher Scientific) according to instructions of a manufacturer. The RNA was analyzed by using the Agilent 2100 Bioanalyzer system (Agilent Biotechnologies). Only a high-quality RNA sample (RNA integrity number ≥7.5) was used to prepare a sample for sequencing. A library was prepared by using the Illumina TruSeq library according to the instructions of the manufacturer. The library was diluted to 2 nM with RSB and Tween 20 and used for sequencing. RNA-seq was performed with a read length of 1×100 bases by using Illumina NextSeq1000 (Illumina) according to the standard Illumina RNA-Seq protocol. Sequence data was evaluated by using NGSQCToolkit v.2.3.3, and an adapter was removed by using Cutadapt v.3.7 with default settings. A low-quality sequence was trimmed by using Sickle v. 1.33 having a Phred quality threshold score of 20, and excluded was a case wherein a trimmed read includes an ambiguous character (e.g., N) or has less than 50 bp. Subsequently, RNA-seq data was processed by using the method of Reference Example 1. Transcripts of 20 blood samples were sequenced by RNA-seq at a total read depth of 39 to 77 M, and 13 to 25 M reads were assigned to an annotated gene GRCm39.

### 4.2. Identification of Dir Transformation in Tumor-induced Mouse

A biomarker including CD and cytokine for a tumor-associated macrophage was characterized to represent a pro-tumor profile of a mouse blood cell. From among mouse CD genes CD68, CD14, CD163, Mrc1, and Msr1 encoding CD biomarkers associated with tumor-associated macrophages CD68, CD14, CD163, CD206, and CD204, three genes CD68, CD14, and Msr1 were characterized as DEGs through analysis. From among the same, appropriate index genes were identified from two DEGs CD68 and Msr1.

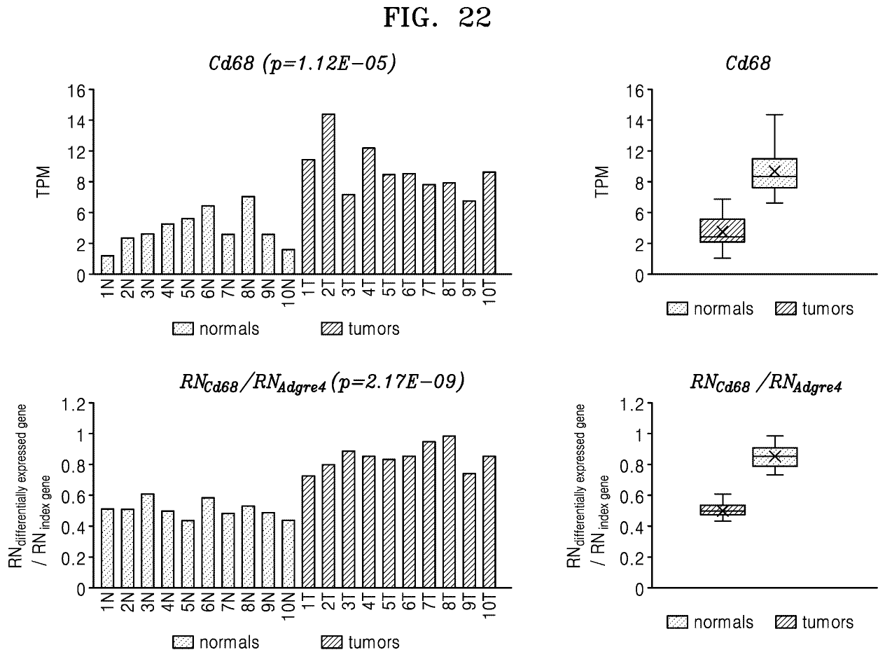

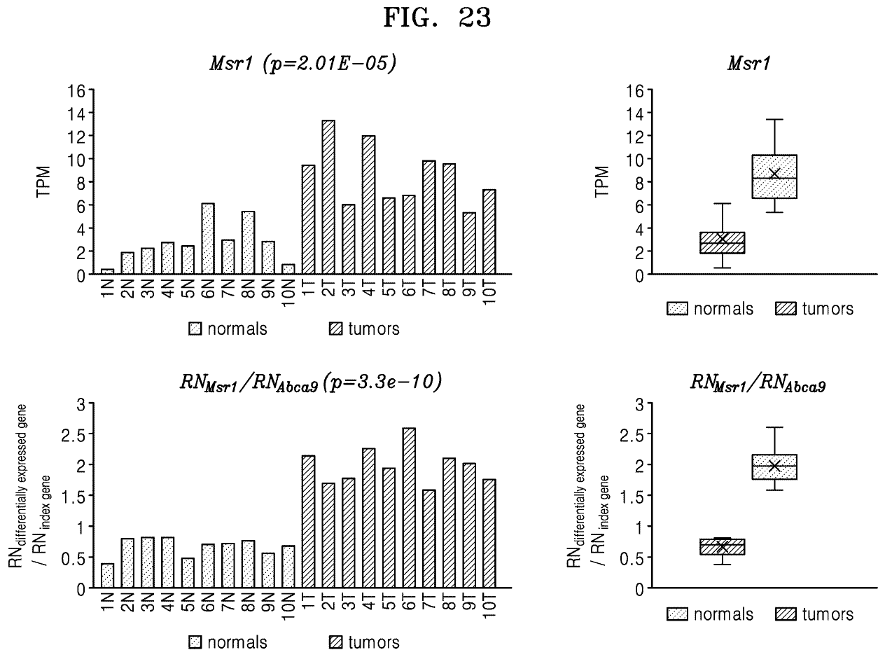

As illustrated in FIGS. 22 and 23, a Dir transformation for RN(CD68)/RN(Adgre4) and RN(Msr1)/RN(Abca9) showed a p-value improvement of 5.17E+03 times and 6.09E+04 times, respectively, compared to TPM, and showed perfect calling accuracy. In addition, the Dir transformations clearly showed a difference between a control mouse and a tumor-induced mouse, compared to TPM.

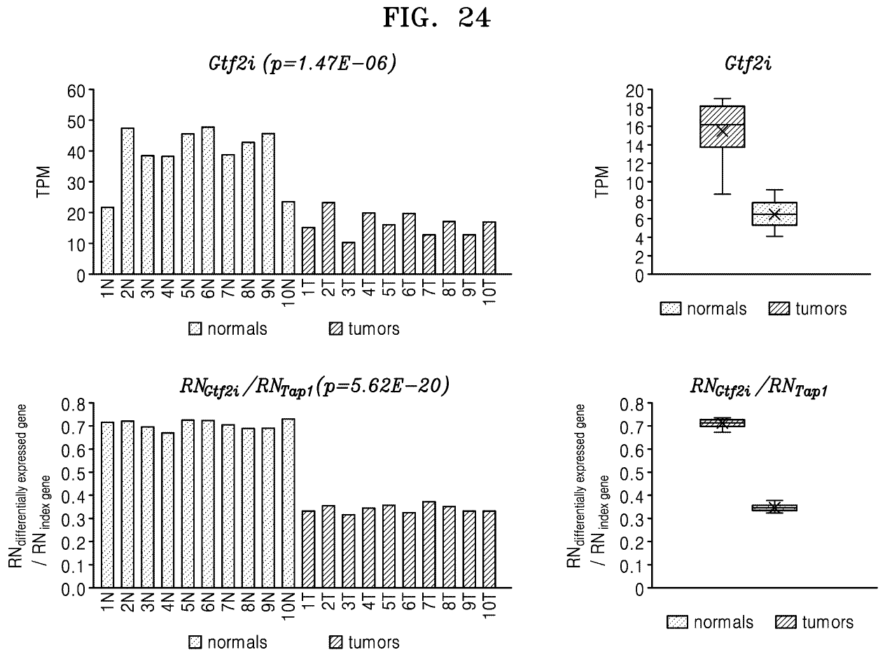

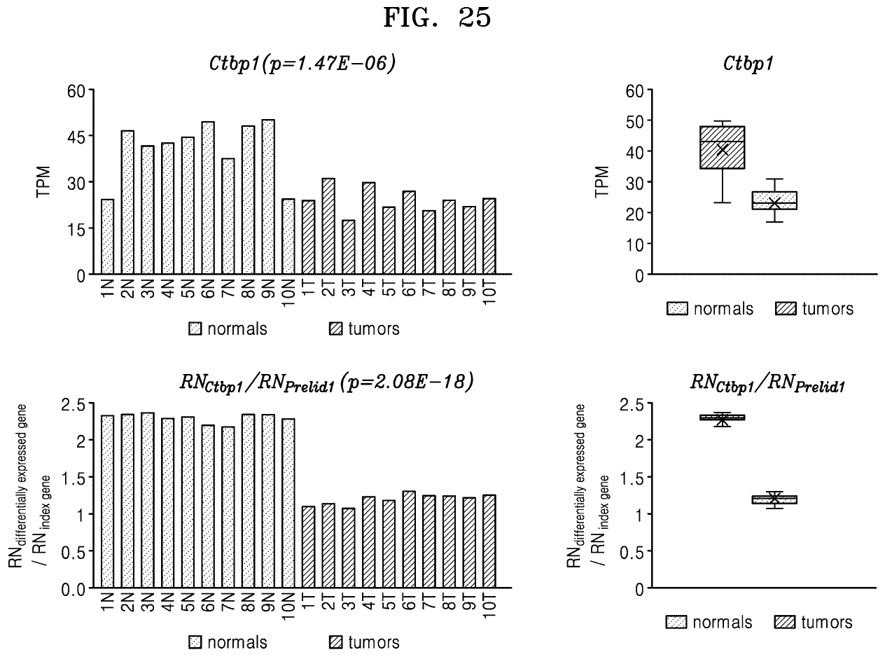

Additionally, a Dir transformation database was constructed to find a better Dir transformation combination. In detail, 8,920 richly expressed genes having an average RN exceeding 50 were selected from 20 RNA-seq data sets. The RN of each gene was divided by the RN of other 8,919 genes, and then NSD of each RN(gene)/RN(other genes) was calculated to classify the lowest NSD. Subsequently, for each gene, 10 genes having the lowest NSD score of the RN (gene)/RN(other genes) were selected. Subsequently, a Dir transformation database was selected under three conditions of an estimated DEG having a p-value less than 0.0001, an index gene having a p-value exceeding 0.05, and ranking according to the lowest Dir transformation p-value. Many Dir transformations using DEGs and index combinations show perfect calling accuracy and greatly low p-values. As illustrated in FIG. 24, RN(Gtf2i)/RN(Tap1) showed the lowest Dir transformation p-value of 5.62E-20. In addition, as illustrated in FIG. 25, RN(Ctbp1)/RN(Prelid1) showed p-value improvement of 3.88E+13 times compared to TPM, and the greatest improvement was identified. Also, two types of Dir transformations produced perfect calling accuracy.

As identified from the above results, normal and disease states may be diagnosed through a Dir transformation.

### Example 5. Elimination of Technical Variations

In the studies of the Drosophila defense system, human prostate cancer, and tumor-induced mouse performed in Embodiments 1 to 4, RNA-seq having a great total mapping RN was used to remove technical variations from analysis. Environmental variations were limitedly identified in all of three types of RNA-seq analysis. Drosophila and a mouse were maintained under well-maintained laboratory conditions to reduce available environmental variations, and in the case of prostate cancer RNA-seq, a cancer tissue and a normal tissue corresponding to the same were separated from the same patient to indicate that the above two samples arise from the same person with the same environmental variation. Under the limited conditions described above, a Dir transformation is effective in reducing environmental variations, but the Dir transformation needs to be applied to a greater dataset of RNA-seq using natural and highly diverse environmental variations to be applied to blood samples from various subjects, and thus, a method of appropriately regulating additional variations is needed.

Therefore, an experiment was performed by additionally using a monocyte RNA-seq data set of a breast cancer patient. The data set included 80 monocyte RNA-seq data sets obtained from 50 normal people and 30 tumor patients. Transcripts from 80 monocyte samples were assigned to an annotated gene GRCh38.

It was assumed that the data set was collected from a subject having natural environmental variations and some RNA-seq data having a small total mapped RN include technical variations. Therefore, for the removal of technical variations from RNA-seq data, a value of correlation coefficient (r) calculated by the read number of all transcripts between two different types of RNA-seq was used. The values of r of 80 samples (50 normal samples and 30 tumor samples) were used to generate an average r for each RNA-seq. As a result, an RNA-sequence having an average r less than 0.98 and an RNA-sequence having a total mapped RN less than 10 million were removed from additional analysis. An RNA-seq data set estimated to have a technical variation, resulted in an unstable RN of a DEG. Therefore, 12 pieces of unstable data were removed, and 44 normal samples and 24 tumor samples were used for additional analysis.

### Example 6. Application of Dir transformation in Monocyte RNA-seq Dataset from Breast Cancer Patient

A Dir transformation was identified by using a total of 68 data sets from which technical variations were removed through Example 5. In detail, 9,407 richly expressed genes having an average RN exceeding 100 were selected from 68 pieces of RNA-Seq data. After dividing the RN of each gene by the RN of other 9,406 genes, NSD of each RN(gene)/RN(other genes) was calculated and aligned from the lowest NSD. Subsequently, ten genes having the lowest NSD score were selected as candidate index genes. To generate a database of Dir transformation candidates, Dir transformation candidates were selected under the following three conditions: an estimated DEG having p<0.0001, an index gene having p >0.05, and ranking according to the lowest Dir transformation p-value. As a result, when a Dir transformation was performed on 44 normal monocyte samples, the lowest p-value of the Dir transformation was observed in RN(CAND1)/RN(IRAG2). The Dir transformation showed a p-value of 7.38E-12, and showed improvement in calling accuracy. However, environmental variations experienced during single-cell collection in the 44 normal samples were too great to be fully normalized, and a possibility of an index gene being randomly generated was identified.

### Example 7. Environmental Variation Clustering

In Example 6 above, an issue occurs in which an index gene may be randomly generated, and thus, environmental variation clustering was performed on each of 44 normal samples to control environmental variations under limited conditions. In detail, a value of a correlation coefficient r between two types of RNA-seq was calculated by using 100 estimated DEGs having the lowest p-value when comparing 44 normal and 24 tumor samples for environmental variation clustering.

As a result of dividing the 44 normal samples into five clusters from clusters A to E, two samples were identified in the cluster A, three samples were identified in the cluster B, 11 samples were identified in the cluster C, eight samples were identified in the cluster D, and 20 samples were identified in the cluster E. Clustering was grouped according to the order of an SRR number that was an identification number provided by NCBI. From the above results of clustering, a Dir transformation was performed on 11 cluster C samples (MC1 to MC11) and 11 cluster E samples (ME10 to ME20).

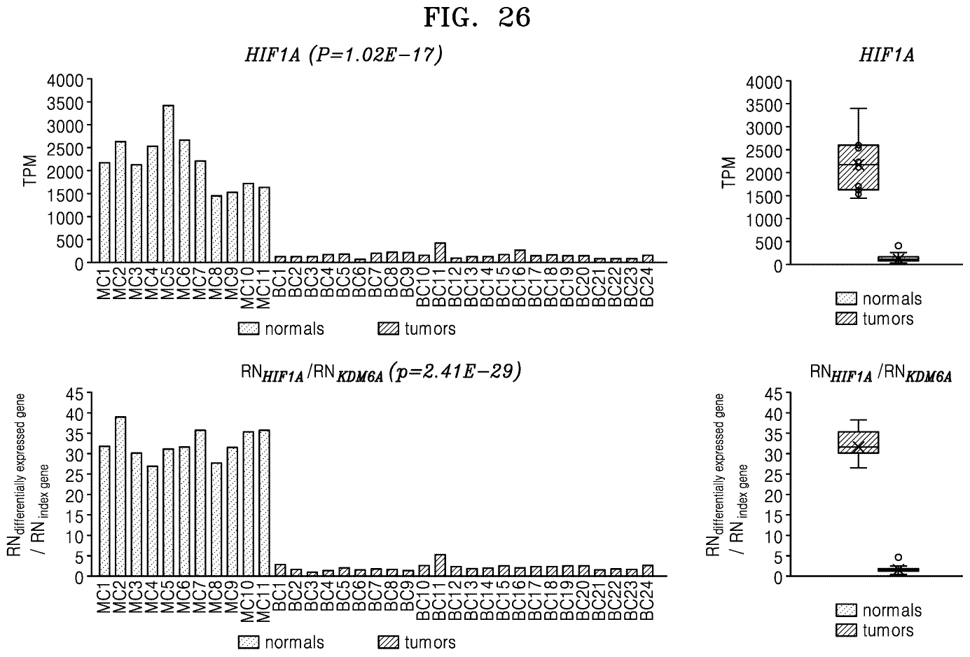

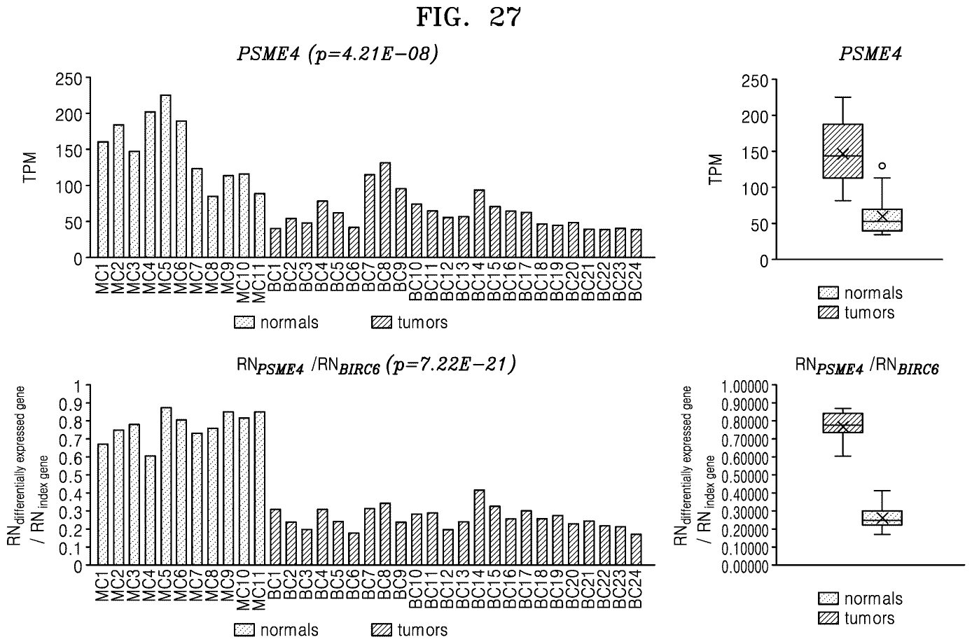

As illustrated in FIG. 26, when a Dir transformation was performed on 11 cluster C samples, the lowest p-value of the Dir transformation was identified to be 2.40E-29 for RN(HIF1A)/RN(KDM6A). As illustrated in FIG. 27, improvement in the greatest p-value of a TPM/Dir transformation was identified to be 5.83E+12 times for RN(PSME4)/RN(BIRC6).

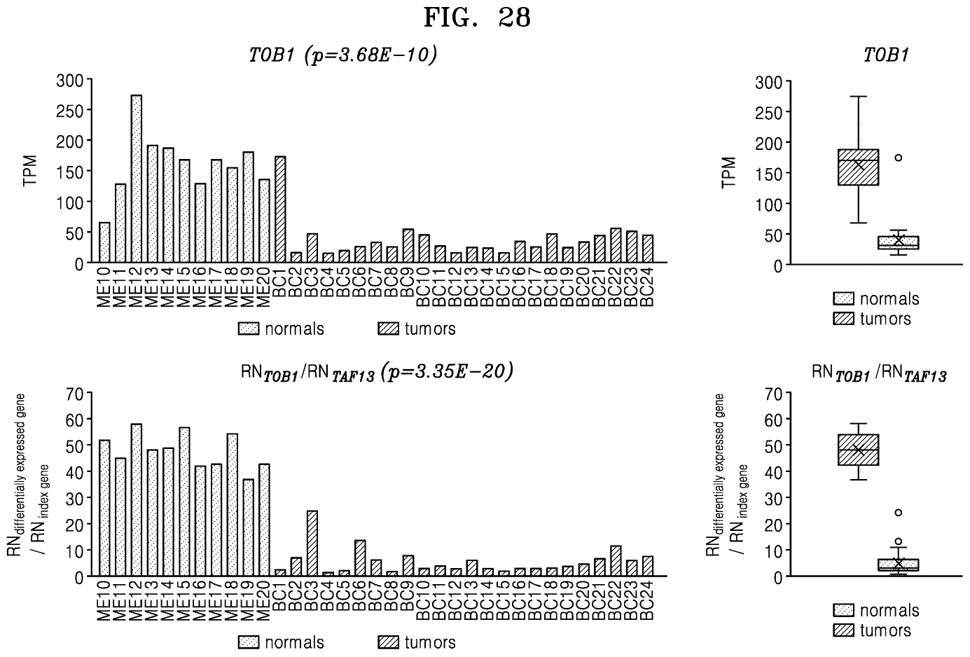

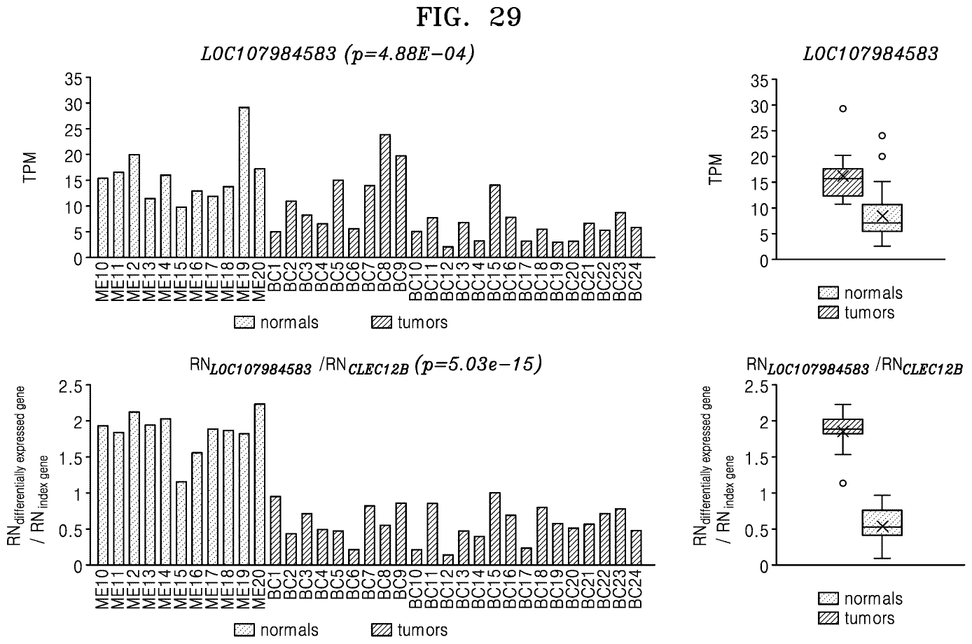

In addition, as illustrated in FIG. 28, the lowest p-value of a Dir transformation was identified to be 3.35E-20 for RN(TOB1)/RN(TAF13). As illustrated in FIG. 29, improvement in the greatest p-value of a TPM/Dir transformation was identified to be 9.70E+10 times for RN(LOC107984583)/RN(CLEC12B).

Accordingly, it was confirmed that not only the Dir transformation but also many other Dir transformations produce all perfect calling accuracy, and completely separate 24 tumor samples from 11 cluster C normal samples.

### Example 8. Comparison of MAGIC Noise Removal Tool and Dir Transformation

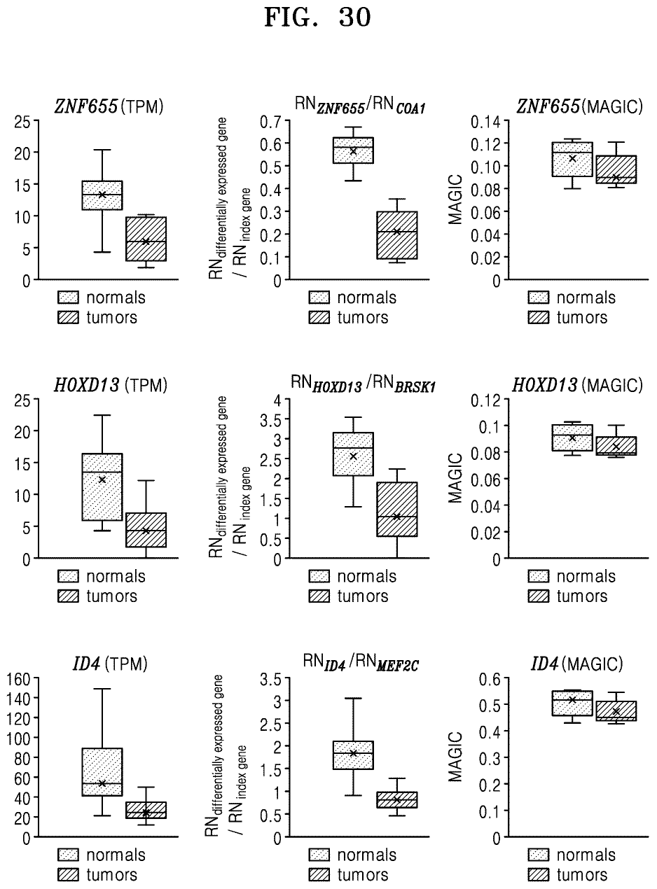

MAGIC was known as a representative noise removal tool that learns an underlying manifold and maps a cell phenotype by using a diffusion operator of scRNA-seq. MAGIC analysis was performed according to the protocol described in Krishnaswamy Lab (https://github.com/KrishnaswamyLab/MAGIC). MAGIC was performed to denoise prostate cancer RNA-seq data, and RNA-seq from which noise was removed by the MAGIC was compared with RNA-seq processed by a Dir transformation. As illustrated in FIG. 30, as a result of removing noise by MAGIC, a distinction between a normal state and a tumor state was not improved in RNA-seq data of prostate cancer. Therefore, it was confirmed that a Dir transformation may be used to diagnose a disease by removing noise and significantly distinguishing between a normal state and a disease state.

### Example 9. Performing of Three-dimensional Dir Transformation

### 9.1. Three-dimensional Dir Transformation Using Index Gene

To rule out an additional error that may appear in a Dir transformation, a three-dimensional Dir transformation was performed by using two index genes. The three-dimensional Dir transformation was performed by using the read number of the two index genes selected with reference to Example 1.1 and the read number of a DEG. The three-dimensional Dir transformation was calculated by using Equation below.

$$T hree - dimensional Dir transformation = \frac{R N ( D E G )}{\sqrt{{( \frac{R N ( index 1 )}{a} )}^{2} + {( \frac{R N ( index 2 )}{b} )}^{2}}}$$

RN(DEG): read number of DEG
RN(index1): read number of first index gene
RN(index2): read number of second index gene
a and b: Relative contribution ratios of respective index genes

In Equation above, in the case where values of a and b, which were the relative contribution ratios of the respective genes, were calculated as 1, the values were calculated by using a simple three-dimensional Dir transformation.

$$T hree - dimensional Dir transformation = \frac{R N ( D E G )}{\sqrt{{( R N ( index 1 ) )}^{2} + {( R N ( index 2 ) )}^{2}}}$$

### 9.2. Identification of Three-dimensional Dir Transformation

To identify the three-dimensional Dir transformation, data of prostate cancer used in Example 2 above was used. In detail, ID4 was used as a DEG, and GNA14 and LOC107985770 were selected and used as index genes. A two-dimensional Dir transformation and a simple three-dimensional Dir transformation were performed.

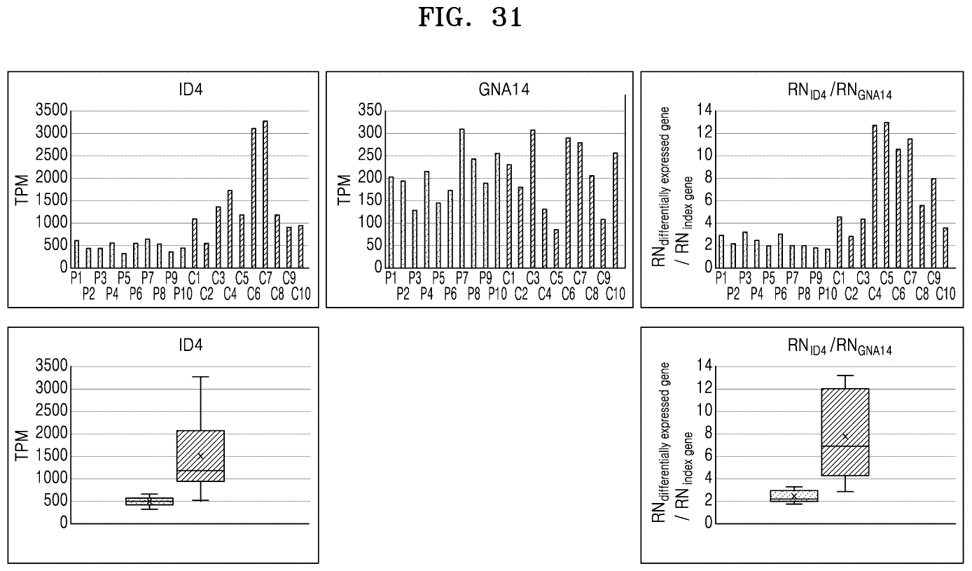

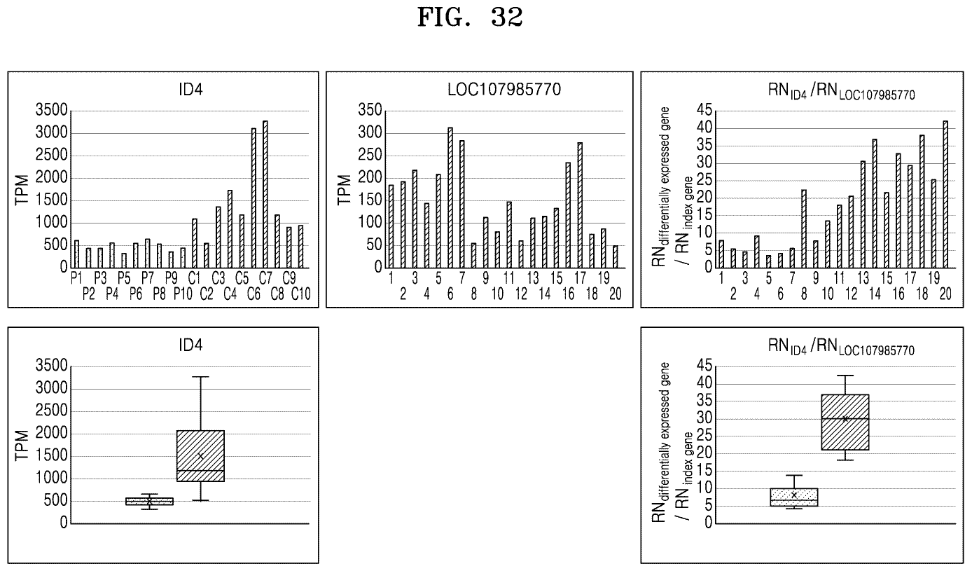

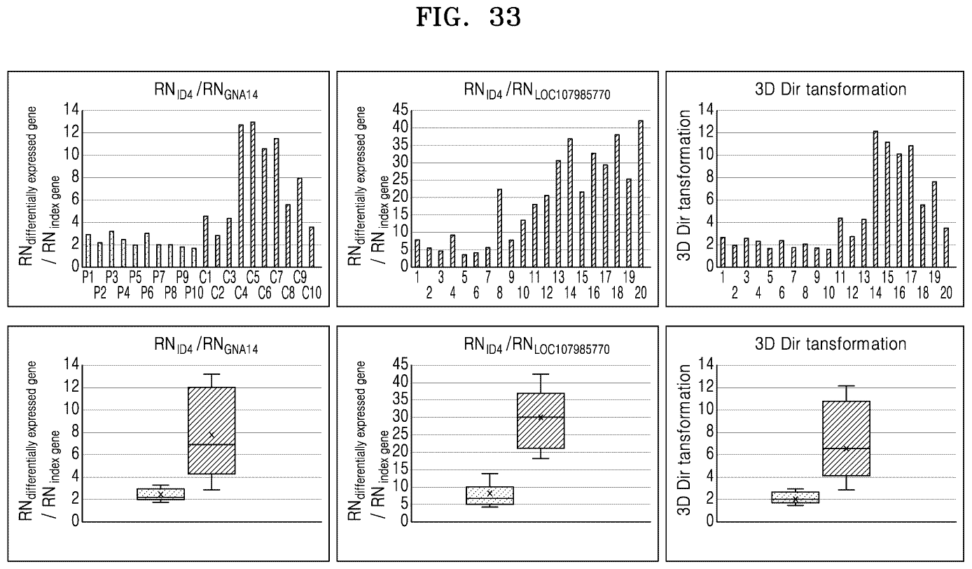

As illustrated in FIGS. 31 and 32, in the case where a two-dimensional Dir transformation was performed, a difference between prostate cancer and a normal tissue was identified not to be great. However, as illustrated in FIG. 33, as a result of performing a simple three-dimensional Dir transformation, a difference between prostate cancer and a normal tissue significantly increases, and an error value identified in a two-dimensional Dir transformation was removed.

As identified from the above results, a probability of a simple three-dimensional Dir transformation was identified, and accordingly, it was confirmed that information for diagnosing a disease may be effectively provided.

1. A method of providing information for diagnosing a disease, the method comprising:
selecting at least one particular index gene from among all genes by using a differentially expressed gene (DEG) differentially expressed in a disease state compared to a normal control group;
performing a multidimensional DEG-index-ratio (Dir) transformation using the DEG and the index gene; and
determining whether or not a difference between DEG expressions of the normal control group and the disease state is significant.

2. The method of claim 1, further comprising providing RNA sequencing data.

3. The method of claim 2, wherein the RNA sequencing data comprises data obtained from a public database or a biological sample.

4. The method of claim 1, wherein the selecting of the particular index gene comprises dividing a read number of the DEG by a read number of all genes and obtaining standard deviation.

5. The method of claim 4, wherein the index gene is determined within at least top 30 candidates when listed in descending order of values of the standard deviation.

6. The method of claim 4, wherein at least two index genes are selected from a candidate group.

7. The method of claim 1, wherein the Dir transformation comprises a two-dimensional Dir transformation, a three-dimensional Dir transformation, or a simple three-dimensional Dir transformation.

8. The method of claim 7, wherein the two-dimensional Dir transformation comprises dividing the read number of the DEG by a read number of an index gene.

9. The method of claim 7, wherein the three-dimensional Dir transformation comprises dividing the read number of the DEG by read numbers of two index genes and relative contribution ratios of respective index genes.

10. The method of claim 7, wherein the simple three-dimensional Dir transformation comprises dividing the read number of the DEG by two index genes.

11. The method of claim 1, wherein the Dir transformation provides information enabling an early diagnosis of a disease by defining a normal range of a disease gene as a narrow range.

12. The method of claim 1, wherein the DEG comprises a transcription factor of an early or late disease gene, or is up-regulated or down-regulated in the disease state.

13. The method of claim 1, wherein the determining of whether or not the difference between the DEG expressions of the normal control group and the disease state is significant comprises determining as the disease state or selecting as a DEG significant for determining to be the disease state in a case where a difference between the normal control group and the DEG is significant, and determining not to be the disease state or selecting a DEG insignificant for determining the disease state in a case wherein the difference is not significant.

14. A method of selecting a gene significant for a disease state, the method comprising:
selecting at least one particular index gene from among all genes by using a differentially expressed gene (DEG) differentially expressed in a disease state compared to a normal control group;
performing a DEG-index-ratio (Dir) transformation using the DEG and the index gene; and
determining whether or not a difference between DEG expressions of the normal control group and the disease state is significant.

15. The method of claim 14, wherein the gene significant for the disease state is used as a biomarker of a disease.

16. The method of claim 14, wherein the gene significant for the disease state is selected from DEGs in which a difference between expressions of the normal control group and the disease state is determined to be significant.

17. A computer-readable recording medium having recorded thereon a program for causing a computer to execute the method of claim 1.
